(19) United States
(12) Patent Application Publication
(10) Pub. No.: US 2015/0225479 A1
(43) Pub. Date: Aug. 13, 2015

(54) Anti-IL-4/Anti-IL-13 Bispecific Antibody/Polyglutamate Formulations

(71) Applicants: Sanofi, Paris (FR); Flamel Ireland Limited, Dublin (IE)

(72) Inventors: Sylvain Huille, Antony (FR); Sophie Carayon, Paris (FR); Corine Vialas, Irigny (FR); Cécile Bonnet-Gonnet, Lyon (FR); You-Ping Chan, Ternay (FR)

(21) Appl. No.: 14/619,975

(22) Filed: Feb. 11, 2015

Related U.S. Application Data
(60) Provisional application No. 61/939,238, filed on Feb. 12, 2014.

(30) Foreign Application Priority Data
Apr. 10, 2014 (EP) 14305524.2

(51) Int. Cl. C07K 16/24; C07K 16/46; A61K 47/22; A61K 47/34; A61K 47/26

(52) U.S. Cl. CPC C07K 16/247; C07K 16/244; A61K 47/34; A61K 47/26; A61K 47/22; C07K 16/468; C07K 2317/76; C07K 2317/31

(57) ABSTRACT

The present invention provides stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, and a cryoprotectant, wherein the formulation has a salt concentration of 50 mM or less. The present invention also provides stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, a cryoprotectant, and a buffering system, wherein the pH of the formulation is about pH 7, and wherein the formulation has a salt concentration of 50 mM or less. The formulations may, optionally, further comprise a surfactant, or a stabilizing agent, or both. The present invention includes methods for making such formulations. The formulations can be used in the treatment of various diseases.

### FIELD OF THE INVENTION

The present invention provides stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, and a cryoprotectant, wherein the formulation has a salt concentration of 50 mM or less. The present invention also provides stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, a cryoprotectant, and a buffering system, wherein the pH of the formulation is about pH 7, and wherein the formulation has a salt concentration of 50 mM or less. The formulations may, optionally, further comprise a surfactant, or a stabilizing agent, or both. The present invention includes methods for making such formulations. The formulations can be used in the treatment of various diseases.

### BACKGROUND OF THE INVENTION

Both IL-4 and IL-13 are therapeutically important cytokines based on their biological functions and play critical roles in many diseases, including asthma (Curr Opin Allergy Clin Immunol 2005, Vo. 5, 161-166). IL-4 has been shown to be able to inhibit autoimmune disease, and IL-4 and IL-13 have both shown the potential to enhance anti-tumor immune responses. Since both cytokines are involved in the pathogenesis of allergic diseases, inhibitors of these cytokines could provide therapeutic benefits.

In order to develop a pharmaceutical formulation containing an anti-IL-4/anti-IL-13 bispecific antibody suitable for subcutaneous administration, the antibody must be concentrated to about 100 mg/mL or greater. However, many complications can arise at such high concentrations, including an increase in viscosity, a shift of pH, a change in the color of the solution, and the formation of visible and sub-visible particles. Formulation of the antibody is further complicated by the fact that it is highly prone to aggregation at high concentrations. While typical antibodies normally form high molecular weight aggregates (HMW) below 5% over a time period of 4 years at 5° C., the anti-IL-4/anti-IL-13 bispecific antibody forms HMW at a rate of between 0.5-1% per hour at 25° C., and at 0.1% per hour at 5° C. Indeed, this antibody has such a strong propensity to aggregate that it cannot be formulated in a liquid in the concentration range targeted. Finally, the anti-IL4/anti-IL13 bispecific antibody has a particularly low isoelectric point, making it more difficult to formulate due to solubility issues.

Prior ready-to-use drug product formulations of the anti-IL-4/anti-IL-13 bispecific antibody comprising standard pharmaceutical excipients had the following composition: antibody 100 mg/mL, Phosphate 6.5 mM/Tris 3.7 mM, pH 7.0, PS80 0.2% (w/v), Sucrose 5% (w/v), and Proline or Mannitol 3% (w/v). These prior formulations are stable, but the antibody still has a strong propensity for aggregation into high molecular weight soluble aggregates over time. Despite comprehensive formulation trials aimed at slowing down aggregation of the antibody, no significant improvements were observed with standard formulation excipients. The instability of the antibody is quite detrimental for the manufacturing process (the formulated drug substance is formulated at slightly less than ⅓ of the final drug product concentration, which should be freeze dried, in order to manage the stability during drug substance/drug product manufacturing) as well as for the in-use stability (extemporaneous reconstitution and injection should be performed in not more than 1 hour at room temperature due to the low stability of antibody at 100 mg/mL). Colloidal aggregation (formation of micronic particles) and chemical degradation are limited in the current formulation. The main degradation pathway is HMW formation, which is closely related to mAb concentration. For example, the free drug substance at 35 mg/mL exhibits +0.9% of HMW after 6 hours and +3.6% of HMW after 24 hours at room temperature, while drug product at 100 mg/mL exhibits +0.6% of HMW after 1 hour and +15% of HMW after 24 hours at room temperature (HMW formation was 10 times slower at 5° C.). Thus, the rate of aggregation of this molecule in the liquid state is a major hurdle for development of a commercial formulation for increasing duration of use after reconstitution and prior to injection, and allowing process scale-up by reducing the constraint on time-out-of-refrigeration (TOR).

Accordingly, a need exists for improved and stable pharmaceutical formulations that can address these complications.

### SUMMARY OF THE INVENTION

An objective of the invention is to provide a formulation in which an anti-IL-4/anti-IL-13 bispecific antibody gains several more hours of stability, as compared to prior formulations of the antibody. Another objective of the invention is to provide a formulation in which aggregates (soluble HMW aggregates) are reduced.

To meet these and other needs, provided herein are highly stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, and a cryoprotectant, wherein the formulation has a salt concentration of 50 mM or less. Also provided herein are highly stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, a cryoprotectant, and a buffering system, wherein the pH of the formulation is about pH 7, and wherein the formulation has a salt concentration of about 50 mM or less. The formulations may, optionally, further comprise a surfactant, or a stabilizing agent, or both. These formulations improve upon conventional formulations, which often lead to aggregation of the antibody upon increasing the concentration of the antibody in the formulation. In particular, the formulations of the invention exhibit good stability regarding high molecular weight proteins.

An embodiment of the invention provides a stable antibody formulation comprising: a bispecific anti-IL-4/anti-IL-13 antibody or an antigen binding fragment thereof, comprising a light chain of the formula VL1-linker-VL2 and a heavy chain of the formula VH1-linker-VH2, wherein VL1 is a variable light chain domain and VH1 is a variable heavy chain domain that form an antigen binding domain for a first antigen (for example, IL-13), and VL2 is a variable light chain domain and VH2 is a variable heavy chain domain that form an antigen binding domain for a second antigen (for example, IL-4); a polyaminoacid consisting of glutamic acid or aspartic acid or both with an average degree of polymerization between 25 and 200, and randomly grafted with 1 to 13% of Vitamin E; and a cryoprotectant; wherein the molar ratio of the antibody versus the polyaminoacid consisting of glutamic acid or aspartic acid or both is between 1:0.25 to 1:2.5, and wherein the formulation contains 50 mM or less of salt.

In other embodiments, the light chain of the bispecific anti-IL-4/anti-IL-13 antibody or an antigen binding fragment thereof comprises the formula N-VL1-linker-VL2-CL-C, wherein CL is a light chain constant domain of an antibody, and the heavy chain of the bispecific anti-IL-4/anti-IL-13 antibody or an antigen binding fragment thereof comprises the formula N-VH1-linker-VH2-CH1-C, wherein CH1 is a first heavy chain constant domain of an antibody. In these embodiments, VL1 is the outer (N-terminal) variable light chain domain. VL1 is linked to VL2. VL2 is the inner (C-terminal) variable light chain domain, which is linked to a constant light chain domain (CL). In these embodiments, VH1 is the outer (N-terminal) variable heavy chain domain. VH1 is linked to VH2. VH2 is the inner (C-terminal) variable light chain domain, which is linked to a constant heavy chain domain (CH1). In these embodiments, VL2 and VH2 form an outer (N-terminal) antigen binding domain, and VL1 and VH1 form an inner (C-terminal) antigen binding domain.

In yet other embodiments, the light chain of the bispecific anti-IL-4/anti-IL-13 antibody comprises the formula N-VL1-linker-VL2-CL-C, wherein CL is a light chain constant domain of an antibody, and the heavy chain of the bispecific anti-IL-4/anti-IL-13 antibody comprises the formula N-VH1-linker-VH2-CH1-CH2-CH3-C, wherein CH1 is a first heavy chain constant domain of an antibody and CH2-CH3 corresponds to the Fc domain of an antibody. In these embodiments, VL1 is the outer (N-terminal) variable light chain domain. VL1 is linked to VL2. VL2 is the inner (C-terminal) variable light chain domain, which is linked to a constant light chain domain (CL). In these embodiments, VH1 is the outer (N-terminal) variable heavy chain domain. VH1 is linked to VH2. VH2 is the inner (C-terminal) variable light chain domain, which is linked to a constant heavy chain domain (CH1). In these embodiments, VL2 and VH2 form an outer (N-terminal) antigen binding domain, and VL1 and VH1 form an inner (C-terminal) antigen binding domain.

In certain embodiments, VL1 comprises the amino acid sequence of SEQ ID NO: 1; VH1 comprises the amino acid sequence of SEQ ID NO: 2; VL2 comprises the amino acid sequence of SEQ ID NO: 3; and VH2 comprises the amino acid sequence of SEQ ID NO: 4 or 5.

In certain embodiments, VL1 comprises the CDR sequences of SEQ ID NO: 1; VH1 comprises the CDR sequences of SEQ ID NO: 2; VL2 comprises the CDR sequences of SEQ ID NO: 3; and VH2 comprises the CDR sequences of SEQ ID NO: 4 or 5.

In one embodiment, VL1 comprises the CDR sequences of RASESVDSYGQSYMH (CDR1; SEQ ID NO: 8), LASNLES (CDR2; SEQ ID NO: 9), and QQNAEDSRT (CDR3; SEQ ID NO: 10), VH1 comprises the CDR sequences of GFSLTDSSIN (CDR1; SEQ ID NO: 11), DGRID (CDR2; SEQ ID NO: 12), and DGYFPYAMDF (CDR3; SEQ ID NO: 13), VL2 comprises the CDR sequences of HASQNIDVWLS (CDR1; SEQ ID NO: 14), KASNLHTG (CDR2; SEQ ID NO: 15), and QQAHSYPFT (CDR3; SEQ ID NO: 16), and VH2 comprises the CDR sequences of GYSFTSYWIH (CDR1; SEQ ID NO: 17), IDPSDGETR (CDR2; SEQ ID NO: 18), and LKEYGNYDSFYFDV (CDR3; SEQ ID NO: 19).

In another embodiment, VL1 comprises the CDR sequences of RASESVDSYGQSYMH (CDR1; SEQ ID NO: 8), LASNLES (CDR2; SEQ ID NO: 9), and QQNAEDSRT (CDR3; SEQ ID NO: 10), VH1 comprises the CDR sequences of GFSLTDSSIN (CDR1; SEQ ID NO: 11), DGRID (CDR2; SEQ ID NO: 12), and DGYFPYAMDF (CDR3; SEQ ID NO: 13), VL2 comprises the CDR sequences of HASQNIDVWLS (CDR1; SEQ ID NO: 14), KASNLHTG (CDR2; SEQ ID NO: 15), and QQAHSYPFT (CDR3; SEQ ID NO: 16), and VH2 comprises the CDR sequences of GYSFTSYWIH (SEQ ID NO: 20), IDASDGETR (SEQ ID NO: 21), and LKEYGNYDSFYFDV (SEQ ID NO: 22).

In certain embodiments, the linker comprises the amino acid sequence of SEQ ID NO: 6.

In certain embodiments, the bispecific antibody or antigen binding fragment thereof further comprises a constant region domain. In specific embodiments, the constant region domain is selected from the group consisting of CH1, CH2, CH3, and CL.

In certain embodiments, the bispecific antibody or antigen binding fragment thereof comprises a constant region domain comprising SEQ ID NO: 23 or a variant thereof. In other embodiments, the bispecific antibody or antigen binding fragment thereof comprises a constant region domain comprising SEQ ID NO: 24 or a variant thereof.

In certain embodiments, the bispecific antibody or antigen binding fragment thereof is a humanized IgG4 bispecific antibody or antigen binding fragment thereof.

In certain embodiments, the concentration of antibody or antigen binding fragment thereof is about 100 mg/mL.

In certain embodiments, the polyaminoacid consisting of glutamic acid or aspartic acid or both has a nominal degree of polymerization between 40 and 120.

In certain embodiments, the polyaminoacid consisting of glutamic acid or aspartic acid or both is randomly grafted with 5-13% of Vitamin E. In specific embodiments, the polyaminoacid consisting of glutamic acid or aspartic acid or both is randomly grafted with 10% of Vitamin E.

In certain embodiments, the polyaminoacid consisting of glutamic acid or aspartic acid or both concentration is about 5-about 10 mg/mL.

In certain embodiments, the cryoprotectant concentration is about 30-about 120 mg/g. In other embodiments, the cryoprotectant concentration is about 40-about 100 mg/g. In further embodiments, the cryoprotectant concentration is about 45-about 90 mg/g.

In certain embodiments, the cryoprotectant is a disaccharide. In specific embodiments, the disaccharide is sucrose. In further specific embodiments, the sucrose concentration is selected from the group consisting of 50 mg/g and 90 mg/g.

In certain embodiments, the aggregate content is not more than 11% after 24 hours at room temperature in liquid form.

In certain embodiments, the formulation is to be used in a liquid form with a concentration of antibody between 75 and 125 mg/ml.

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 10 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 100, and 10% mol/mol of Vitamin E grafted to the polymer; and about 50 mg/g of sucrose; wherein the formulation contains less than 50 mM of salt.

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 10 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 100, and 10% mol/mol of Vitamin E grafted to the polymer; and about 90 mg/g of sucrose; wherein the formulation contains less than 50 mM of salt.

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 5 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 50, and 10% mol/mol of Vitamin E grafted to the polymer; and about 50 mg/g of sucrose; wherein the formulation contains less than 50 mM of salt.

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 5 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 50, and 10% mol/mol of Vitamin E grafted to the polymer; and about 90 mg/g of sucrose; wherein the formulation contains less than 50 mM of salt.

In certain embodiments, the invention includes an antibody formulation comprising: a bispecific anti-IL-4/anti-IL-13 antibody or an antigen binding fragment thereof, comprising a light chain of the formula VL1-linker-VL2 and a heavy chain of the formula VH1-linker-VH2, wherein VL1 is a variable light chain domain and VH1 is a variable heavy chain domain that form an antigen binding domain for a first antigen (for example, IL-13), and VL2 is a variable light chain domain and VH2 is a variable heavy chain domain that form an antigen binding domain for a second antigen (for example, IL-4); a polyaminoacid consisting of glutamic acid or aspartic acid or both with an average degree of polymerization between 25 and 200, and randomly grafted with 1 to 13% of Vitamin E; a cryoprotectant; and a buffering system; wherein the pH of the formulation is about pH 7, and wherein the formulation contains 50 mM or less of salt.

In other embodiments, the light chain of the bispecific anti-IL-4/anti-IL-13 antibody or an antigen binding fragment thereof comprises the formula N-VL1-linker-VL2-CL-C, wherein CL is a light chain constant domain of an antibody, and the heavy chain of the bispecific anti-IL-4/anti-IL-13 antibody or an antigen binding fragment thereof comprises the formula N-VH1-linker-VH2-CH1-C, wherein CH1 is a first heavy chain constant domain of an antibody. In these embodiments, VL1 is the outer (N-terminal) variable light chain domain. VL1 is linked to VL2. VL2 is the inner (C-terminal) variable light chain domain, which is linked to a constant light chain domain (CL). In these embodiments, VH1 is the outer (N-terminal) variable heavy chain domain. VH1 is linked to VH2. VH2 is the inner (C-terminal) variable light chain domain, which is linked to a constant heavy chain domain (CH1). In these embodiments, VL2 and VH2 form an outer (N-terminal) antigen binding domain, and VL1 and VH1 form an inner (C-terminal) antigen binding domain.

In yet other embodiments, the light chain of the bispecific anti-IL-4/anti-IL-13 antibody comprises the formula N-VL1-linker-VL2-CL-C, wherein CL is a light chain constant domain of an antibody, and the heavy chain of the bispecific anti-IL-4/anti-IL-13 antibody comprises the formula N-VH1-linker-VH2-CH1-CH2-CH3-C, wherein CH1 is a first heavy chain constant domain of an antibody and CH2-CH3 corresponds to the Fc domain of an antibody. In these embodiments, VL1 is the outer (N-terminal) variable light chain domain. VL1 is linked to VL2. VL2 is the inner (C-terminal) variable light chain domain, which is linked to a constant light chain domain (CL). In these embodiments, VH1 is the outer (N-terminal) variable heavy chain domain. VH1 is linked to VH2. VH2 is the inner (C-terminal) variable light chain domain, which is linked to a constant heavy chain domain (CH1). In these embodiments, VL2 and VH2 form an outer (N-terminal) antigen binding domain, and VL1 and VH1 form an inner (C-terminal) antigen binding domain.

In certain embodiments, VL1 comprises the amino acid sequence of SEQ ID NO: 1; VH1 comprises the amino acid sequence of SEQ ID NO: 2; VL2 comprises the amino acid sequence of SEQ ID NO: 3; and VH2 comprises the amino acid sequence of SEQ ID NO: 4 or 5.

In certain embodiments, VL1 comprises the CDR sequences of SEQ ID NO: 1; VH1 comprises the CDR sequences of SEQ ID NO: 2; VL2 comprises the CDR sequences of SEQ ID NO: 3; and VH2 comprises the CDR sequences of SEQ ID NO: 4 or 5.

In one embodiment, VL1 comprises the CDR sequences of SEQ ID NO: 1; VH1 comprises the CDR sequences of SEQ ID NO: 2; VL2 comprises the CDR sequences of SEQ ID NO: 3; and VH2 comprises the CDR sequences of SEQ ID NO: 4 or 5.

In another embodiment, VL1 comprises the CDR sequences of RASESVDSYGQSYMH (CDR1; SEQ ID NO: 8), LASNLES (CDR2; SEQ ID NO: 9), and QQNAEDSRT (CDR3; SEQ ID NO: 10), VH1 comprises the CDR sequences of GFSLTDSSIN (CDR1; SEQ ID NO: 11), DGRID (CDR2; SEQ ID NO: 12), and DGYFPYAMDF (CDR3; SEQ ID NO: 13), VL2 comprises the CDR sequences of HASQNIDVWLS (CDR1; SEQ ID NO: 14), KASNLHTG (CDR2; SEQ ID NO: 15), and QQAHSYPFT (CDR3; SEQ ID NO: 16), and VH2 comprises the CDR sequences of GYSFTSYWIH (CDR1; SEQ ID NO: 17), IDPSDGETR (CDR2; SEQ ID NO: 18), and LKEYGNYDSFYFDV (CDR3; SEQ ID NO: 19).

In another embodiment, VL1 comprises the CDR sequences of RASESVDSYGQSYMH (CDR1; SEQ ID NO: 8), LASNLES (CDR2; SEQ ID NO: 9), and QQNAEDSRT (CDR3; SEQ ID NO: 10), VH1 comprises the CDR sequences of GFSLTDSSIN (CDR1; SEQ ID NO: 11), DGRID (CDR2; SEQ ID NO: 12), and DGYFPYAMDF (CDR3; SEQ ID NO: 13), VL2 comprises the CDR sequences of HASQNIDVWLS (CDR1; SEQ ID NO: 14), KASNLHTG (CDR2; SEQ ID NO: 15), and QQAHSYPFT (CDR3; SEQ ID NO: 16), and VH2 comprises the CDR sequences of GYSFTSYWIH (SEQ ID NO: 20), IDASDGETR (SEQ ID NO: 21), and LKEYGNYDSFYFDV (SEQ ID NO: 22).

In certain embodiments, the linker comprises the amino acid sequence of SEQ ID NO: 6.

In certain embodiments, the bispecific antibody or antigen binding fragment thereof further comprises a constant region domain. In specific embodiments, the constant region domain is selected from the group consisting of CH1, CH2, CH3, and CL.

In certain embodiments, the bispecific antibody or antigen binding fragment thereof comprises a constant region domain comprising SEQ ID NO: 23 or a variant thereof. In other embodiments, the bispecific antibody or antigen binding fragment thereof comprises a constant region domain comprising SEQ ID NO: 24 or a variant thereof.

In certain embodiments, the bispecific antibody or antigen binding fragment thereof is a humanized IgG4 bispecific antibody or antigen binding fragment thereof.

In certain embodiments, the concentration of antibody or antigen binding fragment thereof is about 100 mg/mL.

In certain embodiments, the polyaminoacid consisting of glutamic acid or aspartic acid or both has a nominal degree of polymerization between 40 and 120. In specific embodiments, the polyaminoacid consisting of glutamic acid or aspartic acid or both has a nominal degree of polymerization of 50. In other specific embodiments, the polyaminoacid consisting of glutamic acid or aspartic acid or both has a nominal degree of polymerization of 100.

In certain embodiments, the polyaminoacid consisting of glutamic acid or aspartic acid or both is randomly grafted with 5-13% of Vitamin E. In specific embodiments, the polyaminoacid consisting of glutamic acid or aspartic acid or both is randomly grafted with 10% of Vitamin E.

In certain embodiments, the polyaminoacid consisting of glutamic acid or aspartic acid or both concentration is about 5-about 10 mg/mL. In specific embodiments, the polyaminoacid consisting of glutamic acid or aspartic acid or both concentration is about 5 mg/mL. In other specific embodiments, the polyaminoacid consisting of glutamic acid or aspartic acid or both concentration is about 10 mg/mL.

In certain embodiments, the cryoprotectant concentration is about 5% (w/v).

In certain embodiments, the cryoprotectant is a disaccharide. In specific embodiments, the disaccharide is sucrose. In specific embodiments, the sucrose concentration is about 5% (w/v).

In certain embodiments, the buffering system comprises at least two buffers. In certain embodiments, the buffering system concentration is about 10 mM. In certain embodiments, the buffering system comprises Tris buffer and Phosphate buffer. In specific embodiments, the Tris buffer concentration is about 3.7 mM. In other specific embodiments, the Phosphate buffer concentration is about 6.3 mM. In further specific embodiments, the Tris buffer concentration is about 3.7 mM and the Phosphate buffer concentration is about 6.3 mM.

In certain embodiments, the formulation further comprises a surfactant. In specific embodiments, the surfactant concentration is about 0.2% (w/v). In certain embodiments, the surfactant is a polysorbate. In specific embodiments, the polysorbate is polysorbate 80. In specific embodiments, the polysorbate 80 concentration is about 0.2% (w/v).

In certain embodiments, the formulation further comprises a stabilizing agent. In specific embodiments, the stabilizing agent concentration is about 2.5-about 3% (w/v). In certain embodiments, the stabilizing agent is either an amino acid or a sugar. In specific embodiments, the amino acid is proline. In other specific embodiments, the sugar is mannitol. In specific embodiments, the proline concentration is about 3% (w/v). In specific embodiments, the mannitol concentration is about 3% (w/v).

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 10 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 100, and 10% mol/mol of Vitamin E grafted to the polymer; about 10 mM of a buffering system, wherein the buffering system comprises a Tris buffer concentration of about 3.7 mM and a Phosphate buffer concentration of about 6.3 mM; about 0.2% (w/v) polysorbate 80; about 5% (w/v) sucrose; and about 3% (w/v) proline; wherein the pH of the formulation is about pH 7; and wherein the formulation contains less than 50 mM of salt.

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 5 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 50, and 10% mol/mol of Vitamin E grafted to the polymer; about 10 mM of a buffering system, wherein the buffering system comprises a Tris buffer concentration of about 3.7 mM and a Phosphate buffer concentration of about 6.3 mM; about 0.2% (w/v) polysorbate 80; about 5% (w/v) sucrose; and about 3% (w/v) proline; wherein the pH of the formulation is about pH 7; and wherein the formulation contains less than 50 mM of salt.

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 10 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 100, and 10% mol/mol of Vitamin E grafted to the polymer; about 10 mM of a buffering system, wherein the buffering system comprises a Tris buffer concentration of about 3.7 mM and a Phosphate buffer concentration of about 6.3 mM; about 0.2% (w/v) polysorbate 80; about 5% (w/v) sucrose; and about 3% (w/v) mannitol; wherein the pH of the formulation is about pH 7; and wherein the formulation contains less than 50 mM of salt.

In a specific embodiment, the formulation comprises: about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3; about 5 mg/mL of a polyaminoacid consisting of glutamic acid or aspartic acid or both randomly grafted with Vitamin E, wherein the polyaminoacid consisting of glutamic acid or aspartic acid or both has a nominal degree of polymerization of 50, and 10% mol/mol of Vitamin E grafted to the polymer; about 10 mM of a buffering system, wherein the buffering system comprises a Tris buffer concentration of about 3.7 mM and a Phosphate buffer concentration of about 6.3 mM; about 0.2% (w/v) polysorbate 80; about 5% (w/v) sucrose; and about 3% (w/v) mannitol; wherein the pH of the formulation is about pH 7; and wherein the formulation contains less than 50 mM of salt.

In certain embodiments, the formulation is a liquid formulation.

In other certain embodiments, the formulation is a lyophilized formulation.

In certain embodiments, the formulation exhibits good stability regarding high molecular weight proteins.

An embodiment of the invention includes a kit comprising a container comprising the formulation of the invention, and instructions for the administration and use of the formulation.

An embodiment of the invention includes a method for treating an allergic disease, cancer, asthma, a disease associated with abnormal production of IL-4 or IL-13 or both, or a disease associated with an elevated TH-2 mediated response comprising administering to a subject in need thereof a formulation of the invention.

An embodiment of the invention includes a method for making a stable 100 mg/mL antibody formulation comprising: adding a solution of about 10 mg of a polyaminoacid consisting of glutamic acid or aspartic acid or both randomly grafted with Vitamin E, wherein the polyaminoacid consisting of glutamic acid or aspartic acid or both has a nominal degree of polymerization of 100, and 10% mol/mol of Vitamin E grafted to the polymer, to about 100 mg of lyophilized antibody to form a formulation; and stirring the formulation.

An embodiment of the invention includes a method for making a stable 100 mg/mL antibody formulation comprising: a) adding an antibody solution to a solution of polyaminoacid consisting of glutamic acid or aspartic acid or both randomly grafted with Vitamin E, wherein the polyaminoacid consisting of glutamic acid or aspartic acid or both has a nominal degree of polymerization of 100, and 10% mol/mol of Vitamin E grafted to the polymer; b) stirring the combined solution; c) lyophilizing the combined solution; and d) reconstituting the lyophilized powder in water in order to achieve an antibody concentration of 100 mg/mL. In certain embodiments, the method further comprises the step of: adding sucrose to the combined solution in step a).

### BRIEF DESCRIPTION OF THE DRAWINGS

FIG. 1 is a schematic representation of the bispecific anti-IL-4/anti-IL-13 antibody molecule comprising two light chains and two heavy chains. The two light chains comprise the moiety N-VL_{hB-B13}-linker-VL_{h8D4-8}-CL-C, and the two heavy chains comprise the moiety N-VH_{hB-B13}-linker-VH_{h8D4-8}-CH1-CH2-CH3-C. The linker sequence comprises (G4S)₂ or GGGGSGGGGS (SEQ ID NO: 6). In this representation, VL_{hB-B13} and VH_{hB-B13} form an outer (N-terminal) IL-13 antigen binding domain, and VL_{h8D4-8} and VH_{h8D4-8} form an inner (C-terminal) IL-4 antigen binding domain.

FIG. 2 illustrates the amino acid sequences of an exemplary antibody, i.e., humanized variable domains of B-B13 anti-IL-13 antibody (SEQ ID NOS: 1 and 2) and humanized variable domains of 8D4-8 anti-IL-4 antibody (SEQ ID NOS: 3, 4, and 5). Underline indicates amino acid changes made. Bold indicates the CDR sequences.

FIG. 3 is a representation of the schematic structure of polyglutamic acid in ionized state, randomly grafted with R and with sodium as counter ion.

The grafted moiety R corresponding to alpha-tocopheryl is represented in FIG. 4.

FIG. 5 is a picture showing the Direct Reconstitution Process in which PGA polymer solution was added onto lyophilized drug product and stirred for 10 minutes at room temperature on a roller stirrer. This figure corresponds to Examples 1-8.

FIG. 6 is a picture showing the Liquid-Liquid Formulation and Lyophilisation Process in which Antibody solution was slowly added onto PGA polymer solution, freeze-dried, and then reconstituted. This figure corresponds to Examples 9 and 10.

FIG. 7 is a picture showing how the process of FIG. 6 is used to make a formulation of Antibody with P2 polymer and no added salt. This figure corresponds to Examples 14, 15, 17, and 19.

FIG. 8 is a graph showing the aggregation rate of 100 mg/ml of Antibody alone (diamonds) versus 100 mg/ml of Antibody associated with P5 polymer (squares) in a 1:1 molar ratio. This figure corresponds to Example 14.

FIG. 9 is a graph showing the aggregation rate of Antibody alone and Antibody/P2 polymer formulations with no added salt measured by different entities. Diamonds (Example 18) represent the no added salt Antibody alone formulation data obtained by entity B. Squares represent the no added salt Antibody/P2 polymer formulation data obtained by entity A. Triangles (Example 19) represent the no added salt Antibody/P2 polymer formulation data obtained by entity B.

### DETAILED DESCRIPTION

This invention is not limited to the particular methodology, protocols, cell lines, vectors, or reagents described herein because they may vary without departing from the spirit and scope of the invention. Further, the terminology used herein is for the purpose of exemplifying particular embodiments only and is not intended to limit the scope of the present invention. Any method and material similar or equivalent to those described herein can be used in the practice of the present invention and only exemplary methods, devices, and materials are described herein.

All patents and publications mentioned herein are incorporated herein in entirety by reference for the purpose of describing and disclosing the proteins, enzymes, vectors, host cells and methodologies reported therein that might be used with and in the present invention. However, nothing herein is to be construed as an admission that the invention is not entitled to antedate such disclosure by virtue of prior invention.

### A. Definitions

Unless defined otherwise, all technical and scientific terms used herein have the same meaning as is commonly understood by one of ordinary skill in the art.

It is noted here that as used in this specification and the appended claims, the singular forms “a”, “an”, and “the” also include plural reference, unless the context clearly dictates otherwise.

The term “about” or “approximately” means within 10%, for example, within 5% (or 1% or less) of a given value or range.

The terms “administer” or “administration” refers to the act of injecting or otherwise physically delivering a substance as it exists outside the body (e.g., a formulation of the invention) into a patient, such as by mucosal, intradermal, intravenous, subcutaneous, intramuscular delivery or any other method of physical delivery described herein or known in the art. When a disease, or a symptom thereof, is being treated, administration of the substance typically occurs after the onset of the disease or symptoms thereof. When a disease or its symptoms are being prevented, administration of the substance typically occurs before the onset of the disease or symptoms thereof.

In the context of a polypeptide, the term “analog” refers to a polypeptide that possesses a similar or identical function as an anti-IL-4/anti-IL-13 bispecific polypeptide, a fragment of an anti-IL-4/anti-IL-13 bispecific polypeptide, an anti-IL-4/anti-IL-13 bispecific epitope, or an anti-IL-4/anti-IL-13 bispecific antibody, but does not necessarily comprise a similar or identical amino acid sequence of an anti-IL-4/anti-IL-13 bispecific polypeptide, a fragment of an anti-IL-4/anti-IL-13 bispecific polypeptide, an anti-IL-4/anti-IL-13 bispecific epitope, or an anti-IL-4/anti-IL-13 bispecific antibody, or possess a similar or identical structure of an anti-IL-4/anti-IL-13 bispecific polypeptide, a fragment of an anti-IL-4/anti-IL-13 bispecific polypeptide, an anti-IL-4/anti-IL-13 bispecific epitope, or an anti-IL-4/anti-IL-13 bispecific antibody. A polypeptide that has a similar amino acid sequence refers to a polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an anti-IL-4/anti-IL-13 bispecific polypeptide (e.g., SEQ ID NOs: 1-5), a fragment of an anti-IL-4/anti-IL-13 bispecific polypeptide, an anti-IL-4/anti-IL-13 bispecific epitope, or an anti-IL-4/anti-IL-13 bispecific antibody described herein; (b) a polypeptide encoded by a nucleotide sequence that hybridizes under stringent conditions to a nucleotide sequence encoding an anti-IL-4/anti-IL-13 bispecific polypeptide, a fragment of an anti-IL-4/anti-IL-13 bispecific polypeptide, an anti-IL-4/anti-IL-13 bispecific epitope, or an anti-IL-4/anti-IL-13 bispecific antibody (or VH or VL region thereof) described herein of at least 5 amino acid residues, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues (see, e.g., Sambrook et al. (2001) Molecular Cloning: A Laboratory Manual, Cold Spring Harbor Laboratory Press, Cold Spring Harbor, N.Y.; Maniatis et al. (1982) Molecular Cloning: A Laboratory Manual, Cold Spring Harbor Press, Cold Spring Harbor, N.Y.); and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the nucleotide sequence encoding an anti-IL-4/anti-IL-13 bispecific polypeptide, a fragment of an anti-IL-4/anti-IL-13 bispecific polypeptide, an anti-IL-4/anti-IL-13 bispecific epitope, or an anti-IL-4/anti-IL-13 bispecific antibody (or VH or VL region thereof) described herein. A polypeptide with similar structure to an anti-IL-4/anti-IL-13 bispecific antibody polypeptide, a fragment of an anti-IL-4/anti-IL-13 bispecific polypeptide, an anti-IL-4/anti-IL-13 bispecific epitope, or an anti-IL-4/anti-IL-13 bispecific antibody refers to a polypeptide that has a similar secondary, tertiary or quaternary structure of an anti-IL-4/anti-IL-13 bispecific polypeptide, a fragment of an anti-IL-4/anti-IL-13 bispecific polypeptide, an anti-IL-4/anti-IL-13 bispecific epitope, or an anti-IL-4/anti-IL-13 bispecific antibody. The structure of a polypeptide can determined by methods known to those skilled in the art, including but not limited to, X-ray crystallography, nuclear magnetic resonance, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences (e.g., amino acid sequences or nucleic acid sequences) can also be accomplished using a mathematical algorithm. A non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules of interest. BLAST protein searches can be performed with the XBLAST program parameters set, e.g., to score 50, wordlength=3 to obtain amino acid sequences homologous to a protein molecule of interest. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web at ncbi dot nlm dot nih dot gov). Another non-limiting example of a mathematical algorithm utilized for the comparison of sequences is the algorithm of Myers and Miller, 1988, CABIOS 4:11 17. Such an algorithm is incorporated in the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

An “antagonist” or “inhibitor” refers to a molecule capable of inhibiting one or more biological activities of a target molecule, such as signaling by IL-4 or IL-13 or both. Antagonists may interfere with the binding of a receptor to a ligand and vice versa, by incapacitating or killing cells activated by a ligand, or by interfering with receptor or ligand activation (e.g., tyrosine kinase activation) or signal transduction after ligand binding to a receptor. The antagonist may completely block receptor-ligand interactions or may substantially reduce such interactions. In certain embodiments of the invention, the anti-IL-4/anti-IL-13 bispecific antibodies are humanized, antagonistic anti-IL-4/anti-IL-13 bispecific antibodies, such as humanized, monoclonal, antagonistic anti-IL-4/anti-IL-13 bispecific antibodies.

The terms “antibody”, “immunoglobulin”, or “Ig” may be used interchangeably herein. The term antibody includes, but is not limited to, synthetic antibodies, monoclonal antibodies, recombinantly produced antibodies, multispecific antibodies (including bi-specific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above. In particular, antibodies include immunoglobulin molecules and immunologically active portions of immunoglobulin molecules, i.e., antigen binding domains or molecules that contain an antigen-binding site that specifically binds to an IL-4 or IL-13 antigen (e.g., one or more complementarity determining regions (CDRs) of an anti-IL-4/anti-IL-13 bispecific antibody). The anti-IL-4/anti-IL-13 bispecific antibodies can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. In some embodiments, the anti-IL-4/anti-IL-13 bispecific antibodies are humanized, such as humanized monoclonal anti-IL-4/anti-IL-13 bispecific antibodies. In certain embodiments, the anti-IL-4/anti-IL-13 bispecific antibodies are IgG antibodies, human IgG4 antibodies.

The term “antigen” refers to a molecule or a portion of a molecule capable of being bound by the antibodies of the present invention. An antigen can have one or more than one epitope. Examples of antigens recognized by the antibodies of the present invention include, but are not limited to, serum proteins, e.g., cytokines such as IL-4, IL5, IL9 and IL-13, bioactive peptides, cell surface molecules, e.g., receptors, transporters, ion-channels, viral and bacterial proteins.

The term “antigen binding site” refers to the part of the antibody that comprises the area that specifically binds to and is complementary to part or all of an antigen. Where an antigen is large, an antibody may only bind to a particular part of the antigen, which part is termed on epitope. An antigen binding domain may be provided by one or more antibody variable domains. In some embodiments, an antigen binding domain is made of the association of an antibody light chain variable domain (VL) and an antibody heavy chain variable domain (VH).

The term “binding agent” means any molecule, such as an antibody, a siRNA, a nucleic acid, an aptamer, a protein, or a small molecule organic compound, that binds or specifically binds to IL-4 or IL-13 or both, or a variant or a fragment thereof.

The terms “bispecific antibody” or “bispecific antibodies (BsAbs)” refers to molecules that combine the antigen-binding sites of two antibodies within a single molecule. Thus, a bispecific antibody is able to bind two different antigens simultaneously. Besides applications for diagnostic purposes, BsAbs pave the way for new therapeutic applications by redirecting potent effector systems to diseased areas or by increasing neutralizing or stimulating activities of antibodies. Bispecific antibodies can be monoclonal, and in some embodiments are human or humanized. Methods for making bispecific antibodies are well known in the art.

The term “by-product” includes undesired products, which detract or diminish the proportion of therapeutic/prophylactic binding agent, such as an antibody, in a given formulation. For example, typical by-products include aggregates of the antibody, fragments of the antibody, e.g. produced by degradation of the antibody by deamidation or hydrolysis, or mixtures thereof. Typically, aggregates are complexes that have a molecular weight greater than the monomer antibody. Antibody degradation products may include, for example, fragments of the antibody, for example, brought about by deamidation or hydrolysis. Typically, degradation products are complexes that have a molecular weight less than the monomer antibody. In the case of an IgG antibody, such degradation products are less than about 150 kD.

The terms “composition” and “formulation” are intended to encompass a product containing the specified ingredients (e.g., an anti-IL-4/anti-IL-13 bispecific antibody) in, optionally, the specified amounts, as well as any product which results, directly or indirectly, from the combination of the specified ingredients in, optionally, the specified amounts.

The terms “constant region” or “constant domain” refer to a carboxy terminal portion of the light and heavy chain, which is not directly involved in binding of the antibody to antigen but exhibits various effector functions, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

The term “disorder” refers to any condition that would benefit from treatment with the formulation of the present invention. This includes chronic and acute disorders or diseases including those pathological conditions which predispose the mammal, and in particular humans, to the disorder in question. Non-limiting examples of disorders to be treated herein include cancers, inflammation, autoimmune diseases, infections, cardiovascular diseases, respiratory diseases, neurological diseases and metabolic diseases.

The term “epitope” refers to a localized region on the surface of an antigen, such as an IL-4 or IL-13 polypeptide or IL-4 or IL-13 polypeptide fragment, that is capable of being bound to one or more antigen binding regions of a binding agent, such as an antibody, and that has antigenic or immunogenic activity in an animal, such as a mammal, such as a human, that is capable of eliciting an immune response. An epitope having immunogenic activity is a portion of a polypeptide that elicits an antibody response in an animal. An epitope having antigenic activity is a portion of a polypeptide to which an antibody specifically binds, as determined by any method well known in the art, for example, such as an immunoassay. Antigenic epitopes need not necessarily be immunogenic. Epitopes usually consist of chemically active surface groupings of molecules, such as amino acids or sugar side chains, and have specific three dimensional structural characteristics, as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, an IL-4 or IL-13 epitope is a three-dimensional surface feature of an IL-4 or IL-13 polypeptide. In other embodiments, an IL-4 or IL-13 epitope is a linear feature of an IL-4 or IL-13 polypeptide. Anti-IL-4/anti-IL-13 bispecific antibodies may specifically bind to an epitope of the denatured form of IL-4 or IL-13, an epitope of the native form of IL-4 or IL-13, or both the denatured form and the native form of IL-4 or IL-13.

The term “excipients” refers to inert substances that are commonly used as a diluent, vehicle, preservative, binder, stabilizing agent, etc. for drugs and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa., which is hereby incorporated by reference in its entirety.

In the context of a peptide or polypeptide, the term “fragment” refers to a peptide or polypeptide that comprises less than the full length amino acid sequence. Such a fragment may arise, for example, from a truncation at the amino terminus, a truncation at the carboxy terminus, or an internal deletion of a residue(s) from the amino acid sequence. Fragments may, for example, result from alternative RNA splicing or from in vivo protease activity. In certain embodiments, hIL-4 or hIL-13 fragments include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of an IL-4 or IL-13 polypeptide or an antibody that specifically binds to an IL-4 or IL-13 polypeptide.

The term “formulation” means both no salt formulations and low salt formulations of the invention, unless the context states otherwise.

The phrases and terms “functional fragment, variant, derivative or analog” and the like, as well as forms thereof, of an antibody or antigen is a compound or molecule having qualitative biological activity in common with a full-length antibody or antigen of interest. For example, a functional fragment or analog of an anti-IL-4 antibody is one which can bind to an IL-4 molecule or one which can prevent or substantially reduce the ability of a ligand, or an agonistic or antagonistic antibody, to bind to IL-4.

The term “heavy chain” when used in reference to an antibody refers to five distinct types, called alpha (α), delta (Δ), epsilon (ε), gamma (γ), and mu (μ), based on the amino acid sequence of the heavy chain constant domain. These distinct types of heavy chains are well known in the art and give rise to five classes of antibodies, IgA, IgD, IgE, IgG, and IgM, respectively, including four subclasses of IgG, namely IgG1, IgG2, IgG3, and IgG4. In some embodiments, the heavy chain is a human heavy chain. In certain embodiments, the heavy chains of the antibody comprise an “outer” or N-terminal variable heavy chain domain linked to an “inner” or C-terminal variable heavy chain domain, which is linked to a constant heavy chain domain (CH1).

The term “light chain” when used in reference to an antibody refers to two distinct types, called kappa (κ) of lambda (λ) based on the amino acid sequence of the constant domains. Light chain amino acid sequences are well known in the art. In some embodiments, the light chain is a human light chain. In certain embodiments, the light chains of the antibody comprise an “outer” or N-terminal variable light chain domain linked to an “inner” or C-terminal variable light chain domain which is linked to a constant light chain domain (CL).

The term “hinge” or “hinge region” refers to the flexible polypeptide comprising the amino acids between the first and second constant domains of an antibody. One set of amino acids suitable for modification include amino acids in the area of the hinge which impact binding of a molecule containing a heavy chain with binding to the F_{c} receptor and internalization of bound antibody. Such amino acids include, in IgG1 molecules, residues from about 233 to about 237 (Glu-Leu-Leu-Gly-Gly); (SEQ ID NO:25) from about 252 to about 256 (Met-Ile-Ser-Arg-Thr) (SEQ ID NO:26) and from about 318 (Glu) to about 331 (Pro), including, for example, Lys₃₂₀, Lys₃₂₂ and Pro₃₂₉.

“Humanized” forms of non-human (e.g., murine) antibodies are chimeric immunoglobulins, immunoglobulin chains or fragments thereof (such as Fᵥ, F_{ab}, F_{ab′}, F_{(ab)2} or other target-binding subsequences of antibodies) which contain sequences derived from non-human immunoglobulin, as compared to a human antibody. In general, the humanized antibody will comprise substantially all of one, and typically two, variable domains, in which all or substantially all of the CDR regions correspond to those of a non-human immunoglobulin and all or substantially all of the FR regions are those of a human immunoglobulin template sequence. The humanized antibody may also comprise at least a portion of an immunoglobulin constant region (F_{c}), typically that of the human immunoglobulin template chosen. In general, the goal is to have an antibody molecule that is minimally immunogenic in a human. Thus, it is possible that one or more amino acids in one or more CDRs also can be changed to one that is less immunogenic to a human host, without substantially minimizing the specific binding function of the one or more CDRs to IL-4 or IL-13 or both. Alternatively, the FR can be non-human but those amino acids most immunogenic are replaced with ones less immunogenic. Nevertheless, CDR grafting, as discussed above, is not the only way to obtain a humanized antibody. For example, modifying just the CDR regions may be insufficient as it is not uncommon for framework residues to have a role in determining the three-dimensional structure of the CDR loops and the overall affinity of the antibody for its ligand. Hence, any means can be practiced so that the non-human parent antibody molecule is modified to be one that is less immunogenic to a human, and global sequence identity with a human antibody is not always a necessity. So, humanization also can be achieved, for example, by the mere substitution of just a few residues, particularly those which are exposed on the antibody molecule and not buried within the molecule, and hence, not readily accessible to the host immune system. Such a method is taught herein with respect to substituting “mobile” or “flexible” residues on the antibody molecule, the goal being to reduce or dampen the immunogenicity of the resultant molecule without comprising the specificity of the antibody for its epitope or determinant. See, for example, Studnicka et al., Prot Eng 7(6)805-814, 1994; Mol Imm 44:1986-1988, 2007; Sims et al., J Immunol 151:2296 (1993); Chothia et al., J Mol Biot 196:901 (1987); Carter et al., Proc Natl Acad Sci USA 89:4285 (1992); Presta et al., J Immunol 151:2623 (1993), WO 2006/042333 and U.S. Pat. No. 5,869,619.

A humanization method of interest is based on the impact of the molecular flexibility of the antibody during and at immune recognition. Protein flexibility is related to the molecular motion of the protein molecule. Protein flexibility is the ability of a whole protein, a part of a protein or a single amino acid residue to adopt an ensemble of conformations which differ significantly from each other. Information about protein flexibility can be obtained by performing protein X-ray crystallography experiments (see, for example, Kundu et al. 2002, Biophys J 83:723-732.), nuclear magnetic resonance experiments (see, for example, Freedberg et al., J Am Chem Soc 1998, 120(31):7916-7923) or by running molecular dynamics (MD) simulations. An MD simulation of a protein is done on a computer and allows one to determine the motion of all protein atoms over a period of time by calculating the physical interactions of the atoms with each other. The output of a MD simulation is the trajectory of the studied protein over the period of time of the simulation. The trajectory is an ensemble of protein conformations, also called snapshots, which are periodically sampled over the period of the simulation, e.g., every 1 picosecond (ps). It is by analyzing the ensemble of snapshots that one can quantify the flexibility of the protein amino acid residues. Thus, a flexible residue is one which adopts an ensemble of different conformations in the context of the polypeptide within which that residue resides. MD methods are known in the art, see, e.g., Brooks et al. “Proteins: A Theoretical Perspective of Dynamics, Structure and Thermodynamics” (Wiley, New York, 1988). Several software enable MD simulations, such as Amber (see Case et al. (2005) J Comp Chem 26:1668-1688), Charmm (see Brooks et al. (1983) J Comp Chem 4:187-217; and MacKerell et al. (1998) in “The Encyclopedia of Computational Chemistry” vol. 1:271-177, Schleyer et al., eds. Chichester: John Wiley & Sons) or Impact (see Rizzo et al. J Am Chem Soc; 2000; 122(51):12898-12900.)

Most protein complexes share a relatively large and planar buried surface and it has been shown that flexibility of binding partners provides the origin for their plasticity, enabling them to conformationally adapt to each other (Structure (2000) 8, R137-R142). As such, examples of “induced fit” have been shown to play a dominant role in protein-protein interfaces. In addition, there is a steadily increasing body of data showing that proteins actually bind ligands of diverse shapes sizes and composition (Protein Science (2002) 11:184-187) and that the conformational diversity appears to be an essential component of the ability to recognize different partners (Science (2003) 299, 1362-1367). Flexible residues are involved in the binding of protein-protein partners (Structure (2006) 14, 683-693).

The flexible residues can adopt a variety of conformations that provide an ensemble of interaction areas that are likely to be recognized by memory B cells and to trigger an immunogenic response. Thus, antibody can be humanized by modifying a number of residues from the framework so that the ensemble of conformations and of recognition areas displayed by the modified antibody resemble as much as possible those adopted by a human antibody.

That can be achieved by modifying a limited number of residues by: (1) building a homology model of the parent mAb and running an MD simulation; (2) analyzing the flexible residues and identification of the most flexible residues of a non-human antibody molecule, as well as identifying residues or motifs likely to be a source of heterogeneity or of degradation reaction; (3) identifying a human antibody which displays the most similar ensemble of recognition areas as the parent antibody; (4) determining the flexible residues to be mutated, residues or motifs likely to be a source of heterogeneity and degradation are also mutated; and (5) checking for the presence of known T cell or B cell epitopes. The flexible residues can be found using an MD calculation as taught herein using an implicit solvent model, which accounts for the interaction of the water solvent with the protein atoms over the period of time of the simulation. Once the set of flexible residues has been identified within the variable light and heavy chains, a set of human heavy and light chain variable region frameworks that closely resemble that of the antibody of interest are identified. That can be done, for example, using a blast search on the set of flexible residues against a database of antibody human germline sequence. It can also be done by comparing the dynamics of the parent mAb with the dynamics of a library of germline canonical structures. The CDR residues and neighboring residues are excluded from the search to ensure high affinity for the antigen is preserved.

Flexible residues then are replaced. When several human residues show similar homologies, the selection is driven also by the nature of the residues that are likely to affect the solution behavior of the humanized antibody. For instance, polar residues will be in exposed flexible loops over hydrophobic residues. Residues which are a potential source of instability and heterogeneity are also mutated even if there are found in the CDRs. That will include exposed methionines as sulfoxide formation can result from oxygen radicals, proteolytic cleavage of acid labile bonds such as those of the Asp-Pro dipeptide (Drug Dev Res (2004) 61:137-154), deamidation sites found with an exposed asparagine residue followed by a small amino acid, such as Gly, Ser, Ala, His, Asn or Cys (J Chromatog (2006) 837:35-43) and N-glycosylation sites, such as the Asn-X-Ser/Thr site. Typically, exposed methionines will be substituted by a Leu, exposed asparagines will be replaced by a glutamine or by an aspartate, or the subsequent residue will be changed. For the glycosylation site (Asn-X-Ser/Thr), either the Asn or the Ser/Thr residue will be changed.

The resulting composite sequence is checked for the presence of known B cell or linear T-cell epitopes. A search is performed, for example, with the publicly available IEDB. If a known epitope is found within the composite sequence, another set of human sequences is retrieved and substituted.

Unlike the resurfacing method of U.S. Pat. No. 5,639,641, both B-cell-mediated and T-cell-mediated immunogenic responses are addressed by the method. The method also avoids the issue of loss of activity that is sometimes observed with CDR grafting (U.S. Pat. No. 5,530,101). In addition, stability and solubility issues also are considered in the engineering and selection process, resulting in an antibody that is optimized for low immunogenicity, high antigen affinity and improved biophysical properties.

Strategies and methods for resurfacing antibodies, and other methods for reducing immunogenicity of antibodies within a different host, are disclosed, for example, in U.S. Pat. No. 5,639,641. Briefly, in a method, (1) position alignments of a pool of antibody heavy and light chain variable regions are generated to yield heavy and light chain variable region framework surface exposed positions, wherein the alignment positions for all variable regions are at least about 98% identical; (2) a set of heavy and light chain variable region framework surface exposed amino acid residues is defined for a non-human, such as a rodent antibody (or fragment thereof); (3) a set of heavy and light chain variable region framework surface exposed amino acid residues that is most closely identical to the set of rodent surface exposed amino acid residues is identified; and (4) the set of heavy and light chain variable region framework surface exposed amino acid residues defined in step (2) is substituted with the set of heavy and light chain variable region framework surface exposed amino acid residues identified in step (3), except for those amino acid residues that are within 5 Å of any atom of any residue of a CDR of the rodent antibody, to yield a humanized, such as a rodent antibody retaining binding specificity.

Antibodies can be humanized by a variety of other techniques including CDR grafting (EPO 0 239 400; WO 91/09967; and U.S. Pat. Nos. 5,530,101 and 5,585,089), veneering or resurfacing (EPO 0 592 106; EPO 0 519 596; Padlan, 1991, Molec Imm 28(4/5):489-498; Studnicka et al., 1994, Prot Eng 7(6):805-814; and Roguska et al., 1994, PNAS 91:969-973) and chain shuffling (U.S. Pat. No. 5,565,332). Human antibodies can be made by a variety of methods known in the art including, but not limited to, phage display methods, see U.S. Pat. Nos. 4,444,887, 4,716,111, 5,545,806 and 5,814,318; and WO 98/46645, WO 98/50433, WO 98/24893, WO 98/16654, WO 96/34096, WO 96/33735 and WO 91/10741, using transgenic animals, such as rodents, using chimeric cells and so on.

“Interleukin-4” (IL-4) relates to the naturally occurring, or endogenous mammalian IL-4 proteins and to proteins having an amino acid sequence which is the same as that of a naturally occurring or endogenous corresponding mammalian IL-4 protein (e.g., recombinant proteins, synthetic proteins (i.e., produced using the methods of synthetic organic chemistry)). Accordingly, as defined herein, the term includes mature IL-4 protein, polymorphic or allelic variants, and other isoforms of an IL-4 and modified or unmodified forms of the foregoing (e.g., lipidated, glycosylated). Naturally occurring or endogenous IL-4 includes wild type proteins such as mature IL-4, polymorphic or allelic variants and other isoforms and mutant forms which occur naturally in mammals (e.g., humans, non-human primates). Such proteins can be recovered or isolated from a source which naturally produces IL-4, for example. These proteins and proteins having the same amino acid sequence as a naturally occurring or endogenous corresponding IL-4, are referred to by the name of the corresponding mammal. For example, where the corresponding mammal is a human, the protein is designated as a human IL-4. Several mutant IL-4 proteins are known in the art, such as those disclosed in WO 03/038041.

“Interleukin-13” (IL-13) refers to naturally occurring or endogenous mammalian IL-13 proteins and to proteins having an amino acid sequence which is the same as that of a naturally occurring or endogenous corresponding mammalian IL-13 protein (e.g., recombinant proteins, synthetic proteins (i.e., produced using the methods of synthetic organic chemistry)). Accordingly, as defined herein, the term includes mature IL-13 protein, polymorphic or allelic variants, and other isoforms of IL-13 (e.g., produced by alternative splicing or other cellular processes), and modified or unmodified forms of the foregoing (e.g., Hpidated, glycosylated). Naturally occurring or endogenous IL-13 include wild type proteins such as mature IL-13, polymorphic or allelic variants and other isoforms and mutant forms which occur naturally in mammals (e.g., humans, non-human primates). For example, as used herein IL-13 encompasses the human IL-13 variant in which Arg at position 110 of mature human IL-13 is replaced with Gin (position 110 of mature IL-13 corresponds to position 130 of the precursor protein) which is associated with asthma (atopic and nonatopic asthma) and other variants of IL-13. (Heinzmann el al, Hum MoI Genet. 9:549-559 (2000).) Such proteins can be recovered or isolated from a source which naturally produces IL-13, for example. These proteins and proteins having the same amino acid sequence as a naturally occurring or endogenous corresponding IL-13 are referred to by the name of the corresponding mammal. For example, where the corresponding mammal is a human, the protein is designated as a human IL-13. Several mutant IL-13 proteins are known in the art, such as those disclosed in WO 03/035847.

An “isolated” or “purified” binding agent, such as an antibody, is substantially free of cellular material or other contaminating proteins from the cell or tissue source from which the binding agent is derived, or substantially free of chemical precursors or other chemicals when chemically synthesized. For example, the language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also substantially free of culture medium, i.e., culture medium represents less than about 20%, 10%, or 5% of the volume of the protein preparation. When the antibody is produced by chemical synthesis, it is substantially free of chemical precursors or other chemicals, i.e., it is separated from chemical precursors or other chemicals that are involved in the synthesis of the protein. Accordingly, such preparations of the antibody have less than about 30%, 20%, 10%, 5% (by dry weight) of chemical precursors or compounds other than the antibody of interest. In an embodiment, anti-IL-4/anti-IL-13 bispecific antibodies are isolated or purified.

The term “Kabat numbering,” and like terms are recognized in the art and refer to a system of numbering amino acid residues that are more variable (i.e. hypervariable) than other amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3. For the light chain variable region, the hypervariable region typically ranges from amino acid positions 24 to 34 for CDR1, amino acid positions 50 to 56 for CDR2, and amino acid positions 89 to 97 for CDR3.

The term “linker” refers to a molecule that connects the antigen binding domains of the antibody. The linker may be any kind of linker molecule. In some embodiments, the linker is a polypeptide. The linkers may be equal or differ from each other between and within the heavy chain polypeptide and the light chain polypeptide. Furthermore, the linker may have a length of 1, 2, 3, 4, 5, 6, 7, 8, 9, 10, 11, 12, 13, 14, 15, 16, 17, 18, 19 or 20 amino acids. A peptide linker unit for the heavy chain domains as for the light chain domains is (G4S)₂, i.e., GGGGSGGGGS (SEQ ID NO: 6). The numbers of linker units of the heavy chain and of the light chain may be equal (symmetrical order) or differ from each other (asymmetrical order). A peptide linker is long enough to provide an adequate degree of flexibility to prevent the antigen binding moieties from interfering with each others activity, for example by steric hindrance, to allow for proper protein folding and, if necessary, to allow the antibody molecules to interact with two or more, possibly widely spaced, receptors on the same cell; yet it is short enough to allow the antibody moieties to remain stable in the cell.

The terms “low salt” and “low salt concentration” mean a relatively low salt concentration of about 50 mM or less, including a salt concentration of 0 or no salt. The salt concentration considered is determined by the amount of added salts or buffers in the formulation, excluding the PGA polymer and the antibody which are not counted as a salt. It is preferable that the buffering system is present in the formulations in a low concentration, i.e. about 50 mM or less, preferably about 25 mM or less and more preferably about 10 mM or less. Alternatively, some preferred embodiments contain no added salt and no added buffer. It is also preferable that no additional salts, such as NaCl, are added to the formulations.

The terms “manage”, “managing”, and “management” refer to the beneficial effects that a subject derives from a therapy (e.g., a prophylactic or therapeutic agent), which does not result in a cure of the infection. In certain embodiments, a subject is administered one or more therapies (e.g., prophylactic or therapeutic agents, such as a formulation of the invention) to “manage” an IL-4 or IL-13-mediated disease (e.g., cancers, inflammation, autoimmune diseases, infections, cardiovascular diseases, respiratory diseases, neurological diseases, and metabolic diseases), one or more symptoms thereof, so as to prevent the progression or worsening of the disease.

The term “monoclonal antibody” refers to an antibody obtained from a population of homogenous or substantially homogeneous antibodies, and each monoclonal antibody will typically recognize a single epitope on the antigen. In some embodiments, a “monoclonal antibody” is an antibody produced by a single hybridoma or other cell. The term “monoclonal” is not limited to any particular method for making the antibody. For example, monoclonal antibodies may be made by the hybridoma method as described in Kohler et al.; Nature, 256:495 (1975) or may be isolated from phage libraries. Other methods for the preparation of clonal cell lines and of monoclonal antibodies expressed thereby are well known in the art (see, for example, Chapter 11 in: Short Protocols in Molecular Biology, (2002) 5th Ed.; Ausubel et al., eds., John Wiley and Sons, New York).

The term “pharmaceutical composition” as used in the present invention refers to formulations of various preparations. The formulations containing therapeutically effective amounts of the antibodies are sterile liquid solutions, liquid suspensions or lyophilized versions and optionally contain stabilizers or excipients.

The term “pharmaceutically acceptable” means being approved by a regulatory agency of the Federal or a state government, or listed in the U.S. Pharmacopeia, European Pharmacopeia or other generally recognized Pharmacopeia for use in animals, and more particularly in humans.

By “pharmaceutically acceptable excipient” is meant any inert substance that is combined with an active molecule, such as a monoclonal antibody, for preparing an agreeable or convenient dosage form. The “pharmaceutically acceptable excipient” is an excipient that is non-toxic to recipients at the dosages and concentrations employed, and is compatible with other ingredients of the formulation comprising the monoclonal antibody.

The terms “prevent”, “preventing”, and “prevention” refer to the total or partial inhibition of the development, recurrence, onset or spread of an IL-4 or IL-13-mediated disease or symptom related thereto, resulting from the administration of a therapy or combination of therapies provided herein (e.g., a combination of prophylactic or therapeutic agents, such as a formulation of the invention).

The term “prophylactic agent” refers to any agent that can totally or partially inhibit the development, recurrence, onset or spread of an IL-4 or IL-13-mediated disease or symptom related thereto in a subject. In certain embodiments, the term “prophylactic agent” refers to a formulation of the invention. In certain other embodiments, the term “prophylactic agent” refers to an agent other than a formulation of the invention. A prophylactic agent is an agent that is known to be useful to or has been or is currently being used to prevent an IL-4 or IL-13-mediated disease or a symptom related thereto, or impede the onset, development, progression or severity of an IL-4 or IL-13-mediated disease or a symptom related thereto. In specific embodiments, the prophylactic agent is a humanized anti-IL-4/anti-IL-13 bispecific antibody.

The phrase “recombinant antibody” includes antibodies that are prepared, expressed, created, or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic or transchromosomal for human immunoglobulin genes (see, e.g., Taylor, L. D. et al. (1992) Nucl. Acids Res. 20:6287-6295) or antibodies prepared, expressed, created, or isolated by any other means that involves splicing of human immunoglobulin gene sequences to other DNA sequences. Such recombinant antibodies can have variable and constant regions derived from immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to germline VH and VL sequences, may not naturally exist within the antibody germline repertoire in vivo.

The term “saccharide” refers to a class of molecules that are derivatives of polyhydric alcohols. Saccharides are commonly referred to as carbohydrates and may contain different amounts of sugar (saccharide) units, e.g., monosaccharides, disaccharides and polysaccharides.

The terms “specifically binds” or “specifically binding” mean specifically binding to an antigen or a fragment thereof and not specifically binding to other antigens. For example, an antibody that specifically binds to an antigen may bind to other peptides or polypeptides with lower affinity, as determined by, e.g., radioimmunoassays (RIA), enzyme-linked immunosorbent assays (ELISA), BIACORE, or other assays known in the art. Antibodies or variants or fragments thereof that specifically bind to an antigen may be cross-reactive with related antigens. In some embodiments, antibodies or variants or fragments thereof that specifically bind to an antigen do not cross-react with other antigens. An antibody or a variant or a fragment thereof that specifically binds to an IL-4 or IL-13 antigen or both can be identified, for example, by immunoassays, BIAcore, or other techniques known to those of skill in the art. Typically a specific or selective reaction will be at least twice background signal or noise, and more typically more than 10 times background. See, e.g., Paul, ed., 1989, Fundamental Immunology Second Edition, Raven Press, New York at pages 332-336 for a discussion regarding antibody specificity.

A “stable” or “stabilized” formulation is one in which the binding agent, such as an antibody, therein essentially retains its physical stability, identity, integrity, chemical stability, or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993), for example. Stability can be measured at a selected temperature and other storage conditions for a selected time period. The stability may be determined by at least one of the methods selected from the group consisting of visual inspection, SDS-PAGE, IEF, HPSEC, RFFIT, and kappa/lambda ELISA. For example, an antibody “retains its physical stability” in a pharmaceutical formulation, if it shows no signs of aggregation, upon visual examination of color or clarity, or as measured by UV light scattering, SDS-PAGE, or by (high pressure) size exclusion chromatography (HPSEC). When using the formulations of the invention, 11% or less, typically 10% or less, typically 9% or less, typically 8% or less, typically 5% or less, typically 4% or less, typically 3% or less, typically 2% or less, and typically 1% or less of the antibodies form aggregates, as measured by HPSEC or any other suitable method for measuring aggregation formation after 24 hours at room temperature. For example, an antibody is considered stable in a particular formulation if the antibody monomer content is about 90%, about 95%, or about 98% after a certain predetermined period of time under certain storage conditions in a particular formulation. Chemical stability can be assessed by detecting and quantifying chemically altered forms of the protein. Chemical alteration may involve size modification (e.g., clipping), which can be evaluated using (HP)SEC, SDS-PAGE, or matrix-assisted laser desorption ionization/time-of-flight mass spectrometry (MALDI/TOF MS), for example. Other types of chemical alteration include charge alteration (e.g., occurring as a result of deamidation), which can be evaluated by ion-exchange chromatography, for example. An antibody “retains its biological activity” in a pharmaceutical formulation at a given time, if the biological activity of the antibody at a given time is at least about 90% (within the errors of the assay) of the biological activity exhibited at the time the pharmaceutical formulation was prepared, as determined in an antigen binding assay or virus neutralizing assay, for example.

The terms “subject” and “patient” are used interchangeably. As used herein, a subject is, in some embodiments, a mammal, such as a non-primate (e.g., cows, pigs, horses, cats, dogs, rats, etc.) or a primate (e.g., monkey and human), such as a human. In one embodiment, the subject is a mammal, such as a human, having an IL-4 or IL-13-mediated disease or both. In another embodiment, the subject is a mammal, such as a human, at risk of developing an IL-4 or IL-13-mediated disease or both.

The phrase “substantially identical” with respect to an antibody chain polypeptide sequence may be construed as an antibody chain exhibiting at least 70%, 80%, 90%, 95% or more sequence identity to the reference polypeptide sequence. The term with respect to a nucleic acid sequence may be construed as a sequence of nucleotides exhibiting at least about 85%, 90%, 95%, or 97% or more sequence identity to the reference nucleic acid sequence.

“Substitutional” variants are those that have at least one amino acid residue in a native sequence removed and replaced with a different amino acid inserted in its place at the same position. The substitutions may be single, where only one amino acid in the molecule is substituted, or may be multiple, where two or more amino acids are substituted in the same molecule. The plural substitutions may be at consecutive sites. Also, one amino acid can be replaced with plural residues, in which case such a variant comprises both a substitution and an insertion. “Insertional” variants are those with one or more amino acids inserted immediately adjacent to an amino acid at a particular position in a native sequence. Immediately adjacent to an amino acid means connected to either the α-carboxyl or α-amino functional group of the amino acid. “Deletional” variants are those with one or more amino acids in the native amino acid sequence removed. Ordinarily, deletional variants will have one or two amino acids deleted in a particular region of the molecule.

The term “therapeutically effective amount” refers to the amount of a therapy (e.g., a formulation of the invention) that is sufficient to reduce or ameliorate the severity or duration of a given disease or a symptom related thereto. This term also encompasses an amount necessary for the reduction or amelioration of the advancement or progression of a given disease, reduction or amelioration of the recurrence, development or onset of a given disease, or to improve or enhance the prophylactic or therapeutic effect(s) of another therapy (e.g., a therapy other than a formulation of the invention). In some embodiments, the therapeutically effective amount of an antibody of the invention provides a local concentration of between about 5 and 20 ng/ml, or between about 10 and 20 ng/ml. In some embodiments, “therapeutically effective amount” as used herein also refers to the amount of an antibody of the invention to achieve a specified result (e.g., inhibition of an IL-4 or IL-13 cytokine or both).

The term “therapeutic agent” refers to any agent that can be used in the treatment, management or amelioration of an IL-4 or IL-13-mediated disease or both or a symptom related thereto. In certain embodiments, the term “therapeutic agent” refers to a formulation of the invention. In certain other embodiments, the term “therapeutic agent” refers to an agent other than a formulation of the invention. A therapeutic agent is an agent that is known to be useful for, or has been or is currently being used for the treatment, management or amelioration of an IL-4 or IL-13-mediated disease or both or one or more symptoms related thereto.

The term “therapy” refers to any protocol, method, or agent that can be used in the prevention, management, treatment, or amelioration of an IL-4 or IL-13-mediated disease or both (e.g., cancers, inflammation, autoimmune diseases, infections, cardiovascular diseases, respiratory diseases, neurological diseases, and metabolic diseases). In certain embodiments, the terms “therapies” and “therapy” refer to a biological therapy, supportive therapy, or other therapies useful in the prevention, management, treatment, or amelioration of an IL-4 or IL-13-mediated disease or both known to one of skill in the art, such as medical personnel.

The terms “treat”, “treatment”, and “treating” refer to the reduction or amelioration of the progression, severity, or duration of an IL-4 or IL-13-mediated disease or both (e.g., cancers, inflammation, autoimmune diseases, infections, cardiovascular diseases, respiratory diseases, neurological diseases, and metabolic diseases) resulting from the administration of one or more therapies (including, but not limited to, the administration of one or more prophylactic or therapeutic agents, such as a formulation of the invention).

The terms “variable region” or “variable domain” refer to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complementarity determining regions (CDRs), while the more highly conserved regions in the variable domain are called framework regions (FR). The CDRs of the light and heavy chains are primarily responsible for the interaction of the antibody with antigen. Numbering of amino acid positions is according to the EU Index, as in Kabat et al. (1991) Sequences of proteins of immunological interest. (U.S. Department of Health and Human Services, Washington, D.C.) 5^{th} ed. (“Kabat et al.”). In some embodiments, the variable region is a human variable region.

### B. Formulations and Formulation Components

As stated previously, the formulations of the invention comprise stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, and a cryoprotectant, wherein the formulation has a salt concentration of 50 mM or less. The formulations of the invention also comprise stable pharmaceutical antibody formulations, including liquid formulations and lyophilized formulations, comprising an anti-IL-4/anti-IL-13 bispecific antibody, a polyaminoacid consisting of glutamic acid or aspartic acid or both randomly grafted with Vitamin E, a cryoprotectant, and a buffering system, wherein the pH of the formulation is about pH 7, and wherein the formulation has a salt concentration of 50 mM or less. The formulations may, optionally, further comprise a surfactant, or a stabilizing agent, or both. The present invention includes methods for making such formulations. The formulations can be used in the treatment of various diseases. The formulations of the invention have been found to provide significant improvements over prior anti-IL-4/anti-IL-13 bispecific antibody formulations, which often lead to aggregation of the antibody upon increasing the concentration of the antibody in the formulation, and the formation of visible and sub-visible particles. In particular, the formulations of the invention exhibit good stability regarding aggregate formation while containing a low amount of PGA polymer.

i. Anti-IL-4/Anti-IL-13 Bispecific Antibodies, and Variants and Fragments Thereof

In certain embodiments, the formulations of the invention include an anti-IL-4/anti-IL-13 bispecific antibody. The bispecific antibody binds or specifically binds to IL-4 or IL-13 or both, or variants or fragments thereof. The IL-4 or IL-13 or both molecules may be from any species. In some embodiments, the IL-4 or IL-13 or both molecules are from a human. The amino acid sequences and protein structures of both IL-4 and IL-13 are well known in the art.

In certain exemplary embodiments, the anti-IL-4/anti-IL-13 bispecific antibody is a humanized antibody, a fully human antibody, or a variant thereof or an antigen-binding fragment thereof. Some anti-IL-4/anti-IL-13 bispecific antibodies prevent binding of IL-4 and IL-13 with their receptors, and inhibit IL-4 and IL-13 biological activity.

In certain embodiments, the anti-IL-4/anti-IL-13 bispecific antibody or antigen binding fragment thereof is an antibody as described in or produced according to, U.S. Pat. No. 8,388,965, which is hereby incorporated by reference in its entirety. Non-limiting examples include the anti-IL-4/anti-IL-13 bispecific antibodies or antigen binding fragments disclosed in Table 6 of U.S. Pat. No. 8,388,965.

In a specific embodiment, the anti-IL-4/anti-IL-13 bispecific antibody or antigen binding fragment thereof comprises a light chain variable region (VL) that binds to IL-13 comprising the amino acid sequence of SEQ ID NO: 1 (Underline indicates amino acid changes made. Bold indicates the CDR). In this embodiment, the VL that binds to IL-13 is the outer variable light chain domain, and comprises the amino acid sequences RASESVDSYGQSYMH (CDR1; SEQ ID NO: 8), LASNLES (CDR2; SEQ ID NO: 9), and QQNAEDSRT (CDR3; SEQ ID NO: 10).

Anti-IL13 hB-B13 VL3 (SEQ ID NO: 1):
DIVLTQSPAS LAVSLGQRAT ISCRASESVD SYGQSYMHWY
QQKAGQPPKL LIYLASNLES GVPARFSGSG SRTDFTLTID
PVQAEDAATY YCQQNAEDSR TFGGGTKLEI K

In a specific embodiment, the anti-IL-4/anti-IL-13 bispecific antibody or antigen binding fragment thereof comprises a heavy chain variable region (VH) that binds to IL-13 comprising the amino acid sequence of SEQ ID NO: 2 (Underline indicates amino acid changes made. Bold indicates the CDR). In this embodiment, the VH that binds to IL-13 is the inner variable light chain domain, and comprises the amino acid sequences HASQNIDVWLS (CDR1; SEQ ID NO: 14), KASNLHTG (CDR2; SEQ ID NO: 15), and QQAHSYPFT (CDR3; SEQ ID NO: 16).

Anti-IL13 hB-B13 VH2 (SEQ ID NO: 2):
EVQLKESGPG LVAPGGSLSI TCTVSGFSLT DSSINWVRQP
PGKGLEWLGM IWGDGRIDYA DALKSRLSIS KDSSKSQVFL
EMTSLRTDDT ATYYCARDGY FPYAMDFWGQ GTSVTVSS

In a specific embodiment, the anti-IL-4/anti-IL-13 bispecific antibody or antigen binding fragment thereof comprises a light chain variable region (VL) that binds to IL-4 comprising the amino acid sequence of SEQ ID NO: 3 (Underline indicates amino acid changes made. Bold indicates the CDR). In this embodiment, the VL that binds to IL-4 is the outer variable heavy chain domain, and comprises the amino acid sequences GFSLTDSSIN (CDR1; SEQ ID NO: 11), DGRID (CDR2; SEQ ID NO: 12), and DGYFPYAMDF (CDR3; SEQ ID NO: 13).

Anti-IL4 h8D4-8 VL1 (SEQ ID NO: 3):
DIQMTQSPAS LSVSVGDTIT LTCHASQNID VWLSWFQQKP
GNIPKLLIYK ASNLHTGVPS RFSGSGSGTG FTLTISSLQP
EDIATYYCQQ AHSYPFTFGG GTKLEIKR

In a specific embodiment, the anti-IL-4/anti-IL-13 bispecific antibody or antigen binding fragment thereof comprises a heavy chain variable region (VH) that binds to IL-4 comprising the amino acid sequence of SEQ ID NO: 4 (Underline indicates amino acid changes made. Bold indicates the CDR). In this embodiment, the VH that binds to IL-4 is the inner variable heavy chain domain, and comprises the amino acid sequences GYSFTSYWIH (CDR1; SEQ ID NO: 17), IDPSDGETR (CDR2; SEQ ID NO: 18), and LKEYGNYDSFYFDV (CDR3; SEQ ID NO: 19).

Anti-IL4 h8D4-8 VH1 (SEQ ID NO: 4):
QVQLQQSGPE LVKPGASVKI SCKASGYSFT SYWIHWIKQR
PGQGLEWIGM IDPSDGETRL NQRFQGRATL TVDESTSTAY
MQLRSPTSED SAVYYCTRLK EYGNYDSFYF DVWGAGTLVT VSSA

In another specific embodiment, the anti-IL-4/anti-IL-13 bispecific antibody or antigen binding fragment thereof comprises a heavy chain variable region (VH) that binds to IL-4 comprising the amino acid sequence of SEQ ID NO: 5 (Underline indicates amino acid changes made. Bold indicates the CDR). In this embodiment, the VH that binds to IL-4 is the inner variable heavy chain domain, and comprises the amino acid sequences GYSFTSYWIH (CDR1; SEQ ID NO: 20), IDASDGETR (CDR2; SEQ ID NO: 21), and LKEYGNYDSFYFDV (CDR3; SEQ ID NO: 22).

Anti-IL4 h8D4-8 VH2 (SEQ ID NO: 5):
QVQLQQSGPE LVKPGASVKI SCKASGYSFT SYWIHWIKQR
PGQGLEWIGM IDASDGETRL NQRFQGRATL TVDESTSTAY
MQLRSPTSED SAVYYCTRLK EYGNYDSFYF DVWGAGTLVT VSSA

In some specific embodiments, the anti-IL-4/anti-IL-13 bispecific antibody or antigen binding fragment thereof comprises a heavy chain variable region that binds to IL-13 comprising the amino acid sequence of SEQ ID NO: 2; and a light chain variable region that binds to IL-13 comprising the amino acid sequence of SEQ ID NO: 1.

In other specific embodiments, the anti-IL-4/anti-IL-13 bispecific antibody or antigen binding fragment thereof comprises a heavy chain variable region that binds to IL-4 comprising the amino acid sequence of SEQ ID NO: 4; and a light chain variable region that binds to IL-4 comprising the amino acid sequence of SEQ ID NO: 3.

In still other specific embodiments, the anti-IL-4/anti-IL-13 bispecific antibody or antigen binding fragment thereof comprises a heavy chain variable region that binds to IL-4 comprising the amino acid sequence of SEQ ID NO: 5; and a light chain variable region that binds to IL-4 comprising the amino acid sequence of SEQ ID NO: 3.

In more specific embodiments, the anti-IL-4/anti-IL-13 bispecific antibody or antigen binding fragment thereof comprises a heavy chain variable region that binds to both IL-13 and IL-4 comprising the amino acid sequences of SEQ ID NOs: 2 and 4, or 2 and 5; and a light chain variable region that binds to both IL-13 and IL-4 comprising the amino acid sequences of SEQ ID NOs: 1 and 3.

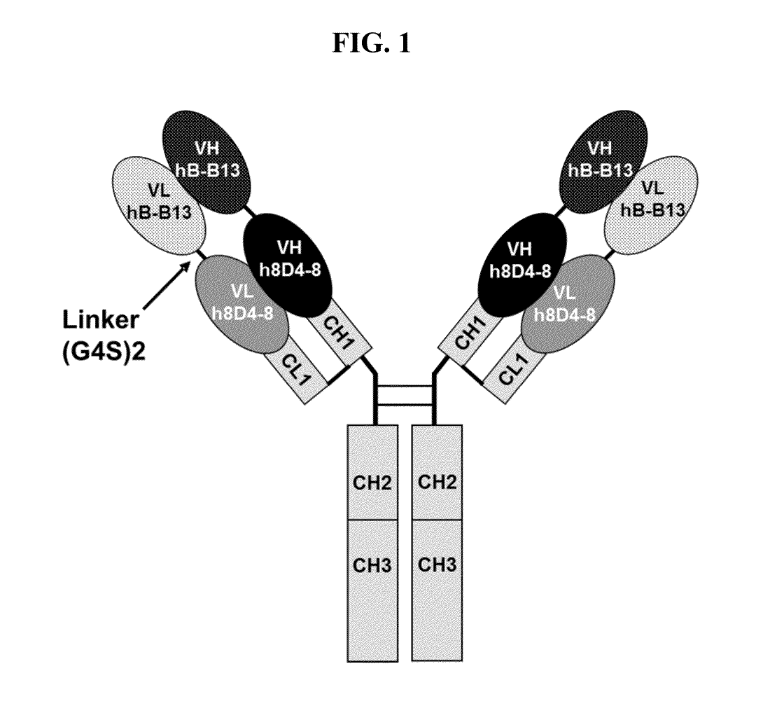

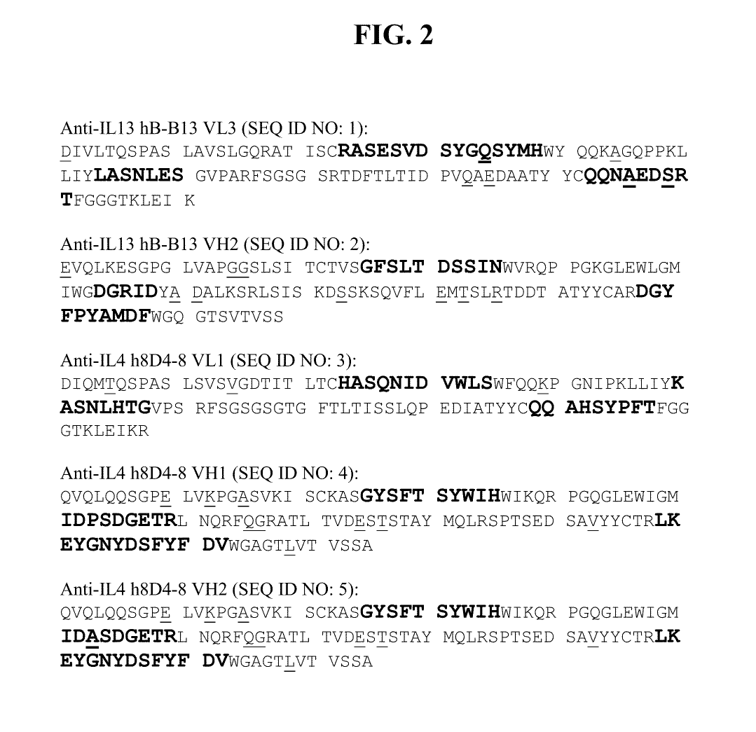

In a most specific embodiment, the anti-IL-4/anti-IL-13 bispecific antibody comprises a heavy chain variable region that binds to both IL-13 and IL-4 comprising the amino acid sequences of SEQ ID NOs: 2 and 4; and a light chain variable region that binds to both IL-13 and IL-4 comprising the amino acid sequences of SEQ ID NOs: 1 and 3 (the “Antibody”). A schematic drawing of an embodiment of the anti-IL-4/anti-IL-13 bispecific antibody is shown in FIG. 1, and exemplary heavy and light chain variable regions are shown in FIG. 2. The molecular weight of the Antibody, as determined by mass spectrometry is 198 kDa (unglycosylated) or 200 kDa (including glycosylation). The isoelectric point of the Antibody, as determined by isoelectric focusing, ranges between 5.8 and 6.2.

In alternative embodiments, the anti-IL-4/anti-IL-13 bispecific antibody or an antigen binding fragment thereof comprises a light chain of the formula VL1-linker-VL2 and a heavy chain of the formula VH1-linker-VH2, wherein VL1 and VH1 form an IL-4 antigen binding domain and VL2 and VH2 form an IL-13 antigen binding domain. In these embodiments, VL2 and VH2 form an outer (N-terminal) antigen binding domain, and the VL1 and VH1 form an inner (C-terminal) antigen binding domain.

In other embodiments, the light chain of the bispecific anti-IL-4/anti-IL-13 antibody or an antigen binding fragment thereof comprises the formula N-VL1-linker-VL2-CL-C, wherein CL is a light chain constant domain of an antibody, and the heavy chain of the bispecific anti-IL-4/anti-IL-13 antibody or an antigen binding fragment thereof comprises the formula N-VH1-linker-VH2-CH1-C, wherein CH1 is a first heavy chain constant domain of an antibody. In these embodiments, VL1 is the outer (N-terminal) variable light chain domain. VL1 is linked to VL2. VL2 is the inner (C-terminal) variable light chain domain, which is linked to a constant light chain domain (CL). In these embodiments, VH1 is the outer (N-terminal) variable heavy chain domain. VH1 is linked to VH2. VH2 is the inner (C-terminal) variable light chain domain, which is linked to a constant heavy chain domain (CH1). In these embodiments, VL2 and VH2 form an outer (N-terminal) antigen binding domain, and VL1 and VH1 form an inner (C-terminal) antigen binding domain.

In yet other embodiments, the light chain of the bispecific anti-IL-4/anti-IL-13 antibody comprises the formula N-VL1-linker-VL2-CL-C, wherein CL is a light chain constant domain of an antibody, and the heavy chain of the bispecific anti-IL-4/anti-IL-13 antibody comprises the formula N-VH1-linker-VH2-CH1-CH2-CH3-C, wherein CH1 is a first heavy chain constant domain of an antibody and CH2-CH3 corresponds to the Fc domain of an antibody. In these embodiments, VL1 is the outer (N-terminal) variable light chain domain. VL1 is linked to VL2. VL2 is the inner (C-terminal) variable light chain domain, which is linked to a constant light chain domain (CL). In these embodiments, VH1 is the outer (N-terminal) variable heavy chain domain. VH1 is linked to VH2. VH2 is the inner (C-terminal) variable light chain domain, which is linked to a constant heavy chain domain (CH1). In these embodiments, VL2 and VH2 form an outer (N-terminal) antigen binding domain, and VL1 and VH1 form an inner (C-terminal) antigen binding domain.

In certain embodiments, VL1 comprises the amino acid sequence of SEQ ID NO: 1; VH1 comprises the amino acid sequence of SEQ ID NO: 2; VL2 comprises the amino acid sequence of SEQ ID NO: 3; and VH2 comprises the amino acid sequence of SEQ ID NO: 4 or 5. In other embodiments, VL2 comprises the amino acid sequence of SEQ ID NO: 1; VH2 comprises the amino acid sequence of SEQ ID NO: 2; VL1 comprises the amino acid sequence of SEQ ID NO: 3; and VH1 comprises the amino acid sequence of SEQ ID NO: 4 or 5.

In certain embodiments, VL1 comprises the CDR sequences of SEQ ID NO: 1; VH1 comprises the CDR sequences of SEQ ID NO: 2; VL2 comprises the CDR sequences of SEQ ID NO: 3; and VH2 comprises the CDR sequences of SEQ ID NO: 4 or 5. In other embodiments, VL2 comprises the CDR sequences of SEQ ID NO: 1; VH2 comprises the CDR sequences of SEQ ID NO: 2; VL1 comprises the CDR sequences of SEQ ID NO: 3; and VH1 comprises the CDR sequences of SEQ ID NO: 4 or 5.

In some embodiments, the anti-IL-4/anti-IL-13 bispecific antibody or antigen binding fragment thereof comprises a linker between the antigen binding domains of the antibody. The linker may be any kind of linker molecule. In some embodiments, the linker is a polypeptide. The linkers may be equal or differ from each other between and within the heavy chain polypeptide and the light chain polypeptide. Furthermore, the linker may have a length of 1, 2, 3, 4, 5, 6, 7, 8, 9, 10, 11, 12, 13, 14, 15, 16, 17, 18, 19 or 20 amino acids. A peptide linker unit for the heavy chain domains as for the light chain domains is (G4S)₂, i.e., GGGGSGGGGS (SEQ ID NO: 6). In specific embodiments, SEQ ID NOs: 2 and 4 are linked together by a first peptide linker, and SEQ ID NOs: 1 and 3 are linked together by a second peptide, wherein the first and second peptide linkers each comprise the amino acid sequence of SEQ ID NO: 6. The numbers of linker units of the heavy chain and of the light chain may be equal (symmetrical order) or differ from each other (asymmetrical order). A peptide linker is long enough to provide an adequate degree of flexibility to prevent the antigen binding moieties from interfering with each others activity, for example by steric hindrance, to allow for proper protein folding and, if necessary, to allow the antibody molecules to interact with two or more, possibly widely spaced, receptors on the same cell; yet it is short enough to allow the antibody moieties to remain stable in the cell. Therefore, the length, composition, or conformation of the linkers joining the tandem variable domains of the bispecific anti-IL-4/anti-IL-13 antibody or an antigen binding fragment thereof can readily be selected by one skilled in the art in order to optimize the desired properties of the polyvalent antibody.

In an embodiment of the invention, the anti-IL-4/anti-IL-13 bispecific antibody or antigen binding fragment thereof is a humanized antibody. Examples of humanized antibody isotypes include IgA, IgD, IgE, IgG, and IgM. In some embodiments, the anti-IL-4/anti-IL-13 bispecific antibody is an IgG antibody. There are four forms of IgG. In some embodiments, the anti-IL-4/anti-IL-13 bispecific antibody is an IgG4 antibody. In some embodiments of the invention, the anti-IL-4/anti-IL-13 bispecific antibody is a humanized IgG4 antibody.

In some embodiments, the anti-IL-4/anti-IL-13 bispecific antibody or antigen binding fragment thereof further comprises a constant region, e.g., CH1, CH2, CH3, and CL.

Certain embodiments of formulations of the invention also include variants of anti-IL-4/anti-IL-13 bispecific antibodies or antigen binding fragments thereof. Variants of anti-IL-4/anti-IL-13 bispecific antibodies may have similar physicochemical properties based on their high similarity, and therefore are also included within the scope of the invention. Variants are defined as antibodies with an amino acid sequence that is at least 95%, at least 97%, for instance at least 98% or 99% homologous to anti-IL-4/anti-IL-13 bispecific antibodies, and capable of competing for binding to an IL-4 or IL-13 polypeptide or both, an IL-4 or IL-13 polypeptide fragment or both, or an IL-4 or IL-13 epitope or both. In some embodiments, the variants will ameliorate, neutralize, or otherwise inhibit IL-4 or IL-13 biological activity or both. Determining competition for binding to the target can be done by routine methods known to the skilled person in the art. In some embodiments, the variants are human or humanized antibodies, such as IgG4 molecules. In some embodiments, a variant is at least 95%, 96%, 97%, 98%, or 99% identical in amino acid sequence with a heavy chain variable region that binds to both IL-13 and IL-4 comprising the amino acid sequences of SEQ ID NOs: 2, 4, and 5; and a light chain variable region that binds to both IL-13 and IL-4 comprising the amino acid sequences of SEQ ID NOs: 1 and 3. The term “variant” refers to an antibody that comprises an amino acid sequence that is altered by one or more amino acids compared to the amino acid sequences of the anti-IL-4/anti-IL-13 bispecific antibody. The variant may have conservative sequence modifications, including amino acid substitutions, modifications, additions, and deletions.

Examples of modifications include, but are not limited to, glycosylation, acetylation, pegylation, phosphorylation, amidation, derivatization by known protecting/blocking groups, proteolytic cleavage, and linkage to a cellular ligand or other protein. Amino acid modifications can be introduced by standard techniques known in the art, such as site-directed mutagenesis, molecular cloning, oligonucleotide-directed mutagenesis, and random PCR-mediated mutagenesis in the nucleic acid encoding the antibodies. Conservative amino acid substitutions include the ones in which the amino acid residue is replaced with an amino acid residue having similar structural or chemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine, tryptophan), nonpolar side chains (e.g., glycine, alanine, valine, leucine, isoleucine, proline, phenylalanine, methionine), beta-branched side chains (e.g., threonine, valine, isoleucine), and aromatic side chains (e.g., tyrosine, phenylalanine, tryptophan). It will be clear to the skilled artisan that classifications of amino acid residue families other than the one used above can also be employed. Furthermore, a variant may have non-conservative amino acid substitutions, e.g., replacement of an amino acid with an amino acid residue having different structural or chemical properties. Similar minor variations may also include amino acid deletions or insertions, or both. Guidance in determining which amino acid residues may be substituted, modified, inserted, or deleted without abolishing immunological activity may be found using computer programs well known in the art. Computer algorithms, such as, inter alia, Gap or Bestfit, which are known to a person skilled in the art, can be used to optimally align amino acid sequences to be compared and to define similar or identical amino acid residues. Variants may have the same or different, either higher or lower, binding affinities compared to an anti-IL-4/anti-IL-13 bispecific antibody, but are still capable of specifically binding to IL-4 or IL-13 or both, and may have the same, higher or lower, biological activity as the anti-IL-4/anti-IL-13 bispecific antibody.

Embodiments of the invention also include antigen binding fragments of the anti-IL-4/anti-IL-13 bispecific antibodies. The term “antigen binding domain,” “antigen binding region,” “antigen binding fragment,” and similar terms refer to that portion of an antibody which comprises the amino acid residues that interact with an antigen and confer on the binding agent its specificity and affinity for the antigen (e.g., the complementary determining regions (CDR)). The antigen binding region can be derived from any animal species, such as rodents (e.g., rabbit, rat or hamster) and humans. In some embodiments, the antigen binding region will be of human origin. Non-limiting examples of antigen binding fragments include: Fab fragments, F(ab′)2 fragments, Fd fragments, Fv fragments, single chain Fv (scFv) molecules, dAb fragments, and minimal recognition units consisting of the amino acid residues that mimic the hypervariable region of the antibody.

In some embodiments of the invention, the anti-IL-4/anti-IL-13 bispecific antibody (or a variant thereof or an antigen binding fragment thereof) will ameliorate, neutralize, or otherwise inhibit IL-4 or IL-13 or both biological activity in vivo.

In some embodiments of the invention, the anti-IL-4/anti-IL-13 bispecific antibodies (or a variant thereof or an antigen binding fragment thereof) are antagonist antibodies that ameliorate, neutralize, or otherwise inhibit IL-4 or IL-13 or both biological activity in vivo.

Identification, isolation, preparation, and characterization of anti-IL-4/anti-IL-13 bispecific antibodies or variants or fragments thereof that bind to both IL-13 and IL-4, including the anti-IL-4/anti-IL-13 bispecific antibody comprising a heavy chain variable region comprising the amino acid sequences of SEQ ID NOs: 2 and 4, and a light chain variable region comprising the amino acid sequences of SEQ ID NOs: 1 and 3, have been described in detail in PCT Publication WO 2009/052081, which is incorporated herein by reference.

In some embodiments, the anti-IL-4/anti-IL-13 bispecific antibodies (or a variant thereof or an antigen binding fragment thereof) are present in the formulations in an amount from about 5 mg/mL to about 200 mg/mL, e.g., about 50 mg/mL to about 150 mg/mL, about 75 mg/mL to about 125 mg/mL, and about 100 mg/mL. Alternatively, the anti-IL-4/anti-IL-13 bispecific antibodies (or a variant thereof or an antigen binding fragment thereof) are present in the formulations in an amount from about 5 mg/mL to about 65 mg/mL, about 66 mg/mL to about 130 mg/mL, about 131 mg/mL to about 200 mg/mL. For example, the anti-IL-4/anti-IL-13 bispecific antibody may be present in the formulation in an amount of about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, or about 200 mg/mL.

In certain exemplary embodiments, the anti-IL-4/anti-IL-13 bispecific antibody is present in the formulation in an amount of about 100 mg/mL. In another exemplary embodiment, a humanized IgG4 anti-IL-4/anti-IL-13 bispecific antibody comprising a heavy chain variable region that binds to both IL-13 and IL-4 comprising the amino acid sequences of SEQ ID NOs: 2 and 4, or 2 and 5; and a light chain variable region that binds to both IL-13 and IL-4 comprising the amino acid sequences of SEQ ID NOs: 1 and 3 is present in the formulation in an amount of about 100 mg/mL.

ii. Poly (Glutamic Acid or Aspartic Acid or Both) Polymers Grafted with Vitamin E (PGA)

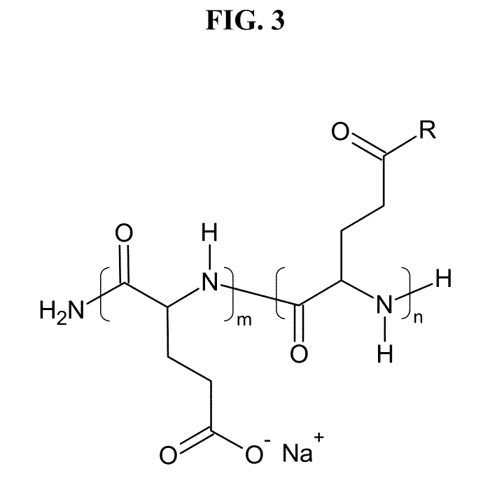

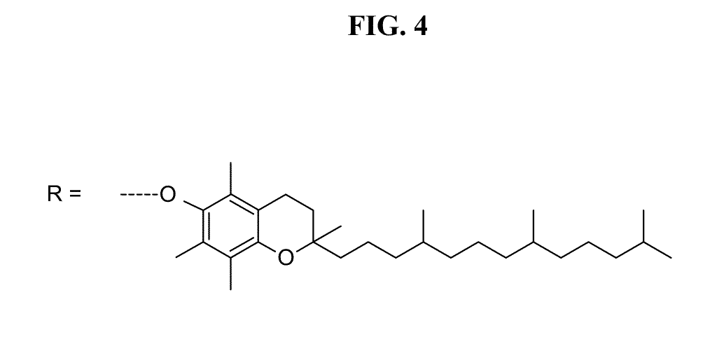

The formulations of the invention include linear poly(glutamic acid or aspartic acid or both) polymers that are randomly grafted with Vitamin E. That is, the PGA polymers have a poly(glutamic acid or aspartic acid or both) backbone, and the Vitamin E moiety is attached to the carboxylic acid group of the glutamate or aspartate side chain via a covalent bond (see FIGS. 3 & 4). These polymers will be referred as PGA polymers hereafter.

In one embodiment, the PGA polymers have a poly (alpha-glutamic acid or alpha-aspartic acid or both) backbone. The PGA polymers in the invention can either be of L, D or racemic (D,L) configuration. In another embodiment, the PGA polymer backbone consists of glutamic acid units.

The term “Vitamin E”, hereafter also designated by VE, refers to a family of compounds possessing a chromanol head with a 16-carbon side chain attached at the 2 position. There are eight naturally occurring forms of Vitamin E, including four tocopherols and four tocotrienols. Tocopherols contain three chiral centers, making eight stereoisomers possible. Tocotrienols, on the other hand, have only one chiral center to allow for two stereoisomers. However, the double bounds of the farnesyl tail allow for the existence of additional four cis/trans isomers per tocotrienol, giving a total of eight isomers possible. See, e.g., GRAS Notification and Exemption Claim for DeltaGold®, a tocotrienol rich extract, GRAS Notice (GRN) No. 471 at page 5-6.

The Vitamin E used in the invention can either be of natural or synthetic origin. In one embodiment of the invention, the Vitamin E consists of (+/−)alpha-tocopherol.

Different PGA polymers can be formed by varying the number of glutamic acid or aspartic acid units in the polymer chain, i.e., the nominal degree of polymerization, and the mol/mol of Vitamin E grafted to the polymer.

The number of glutamic acid units in the polymer chain can range from about 1 to about 225. In some embodiments, the PGA polymer has about 220 glutamic acid units. In other embodiments, the PGA polymer has about 100 glutamic acid units. In yet other embodiments, the PGA polymer has about 50 glutamic acid units.

The mol/mol of Vitamin E grafted to the polymer can range from about 5-about 15%. In some embodiments, the mol/mol of Vitamin E grafted to the polymer is about 5%. In other embodiments, the mol/mol of Vitamin E grafted to the polymer is about 10%. In yet other embodiments, the mol/mol of Vitamin E grafted to the polymer is about 15%. The average molar grafting rate of the poly(glutamic acid) with Vitamin E is the ratio between the average number of monomers grafted with Vitamin E and the total number of monomers.

The PGA polymers contain carboxylic functions that can be either neutral (COOH form) or ionized, depending on the pH. The PGA polymers used in the invention have a major part of their free carboxylic functions in the ionized state. In that state and whatever the pH of the formulation between 4.5 and 12, which may be adjusted by adding for example hydrochloric acid or sodium hydroxide, the PGA polymers self-assemble in aqueous medium to form a stable colloidal solution of nano-sized (around 100 nm or less) particles. In an acidic medium of pH below 4.5 which corresponds to an ionization fraction of COOH groups inferior to 0.05, the PGA polymers remain insoluble. Preferably, the polymer is isolated under its insoluble form at pH<2 at the end of the synthesis. Then the PGA polymers are neutralized by reacting with a strong base (such as sodium hydroxide) in water in presence of a polar solvent (such as ethanol), purified by ultrafiltration using a 1 kDa membrane and concentrated above 30 mg/ml to get a stable colloidal solution. This solution is filtered on a 0.22 μm membrane and stored at 5° C. before use.

The counter ion of the ionized carboxylic functions may be a metallic cation such as for example sodium, potassium, calcium or magnesium or an organic cation such as for example tetramethylammonium, tetrabutylammonium, triethanolamonium, or polyamonium such as for example polyethyleneiminium.

The PGA polymers and formulations of the invention may be made and used according to the methods in U.S. Pat. Nos. 6,630,171 and 7,683,024, each of which is incorporated herein by reference in its entirety.

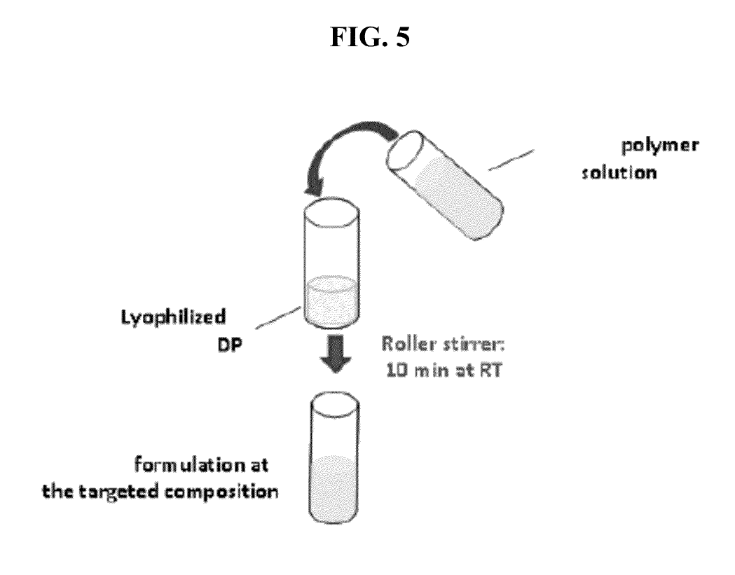

The formulations of the invention may be made by direct reconstitution, that is, PGA polymer solution is added to lyophilized drug product in order to generate the formulation of the invention at the targeted concentration ready for injection. See FIG. 5.

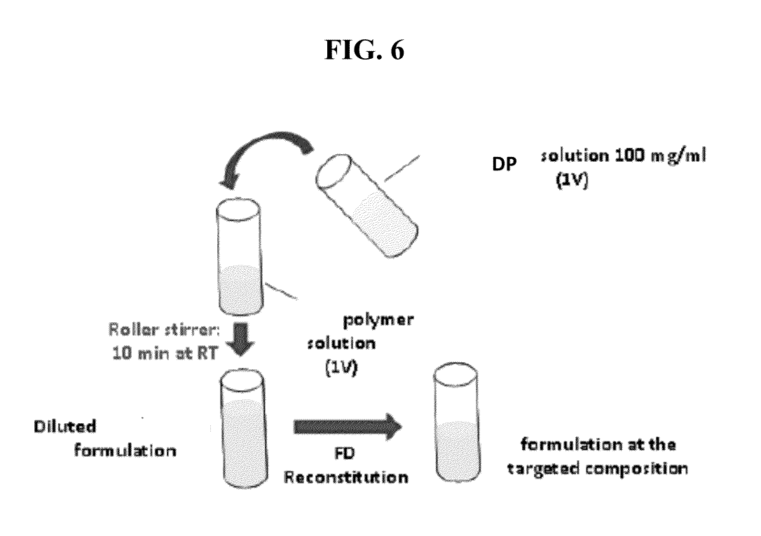

Alternatively, the formulations of the invention may be made by another process called liquid-liquid formulation, lyophilisation and reconstitution. In this method, a solution of drug product is added to a solution of PGA polymer. The combined solution is then lyophilized, and then reconstituted with injectable liquid to the targeted concentration to be injected. See FIG. 6.

In preferred embodiment, the formulations of the invention contains the bispecific antibody and the PGA polymer in a molar ratio comprised between about 1:0.25 to about 1:2.5, preferably about 1:0.5 to about 1:2 and more preferably about 1:1.

iii. Cryoprotectant

The formulations of the invention further comprise a cryoprotectant. Typically, the cryoprotectant can be, for example, a sugar, polyvinylpyrrolidone, or polyethylene glycol. By “sugar” is meant simple sugars (small molecules composed of one or two carbohydrate units) or complex sugars (long chains of carbohydrate units), but also polyols in general. Examples of sugars include, but are not limited to, monosaccharides, disaccharides, and polysaccharides. Examples of saccharides include lactose, maltose, dextrose, glucose, fructose, sucrose, mannitol, xylitol, crythritol, sorbitol, trehalose, and mixtures thereof.

In some embodiments, the cryoprotectant is present in the formulations at a concentration from about 1% to about 10% (w/v). For example, the cryoprotectant may be present in the formulation at a concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

In some embodiments, sugars are used as the cryoprotectant.

In some embodiments, the sugar is present in the formulations at a concentration from about 1% to about 10% (w/v), e.g., about 2% to about 8% (w/v), about 3% to about 7% (w/v), about 4% to about 6% (w/v), or about 5% (w/v). Alternatively, the sugar is present in the formulations at a concentration from about 1% to about 3% (w/v), about 3% to about 6% (w/v), or about 6% to about 10% (w/v). For example, the sugar may be present in the formulations in an amount of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In particular embodiments, the sugar is present in the formulations from about 3% to about 7% (w/v), and, in some embodiments, about 5%.

Alternatively, the sugar is present in the formulations at a concentration of about 80 to about 120 mg/ml. Alternatively, the sugar is present in the formulations at a concentration of about 40 to about 60 mg/ml.

Those skilled in the art are aware that other sugars can be used as long as they are pharmaceutically acceptable, i.e. suitable for administration to subjects. In specific embodiments, the sugar is sucrose.

In certain embodiments, sucrose is present in the formulations in an amount from about 1% to about 10% (w/v). For example, sucrose may be present in the formulation in an amount of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, sucrose may be present in an amount of about 3% to about 7% (w/v), or about 4% to about 6% (w/v). In specific embodiments, sucrose is present in the formulations in an amount of about 5% (w/v).

Alternatively, sucrose is present in the formulations at a concentration of about 80 to about 120 mg/g, or at a concentration of about 10-about 20 mg/ml. Alternatively, sucrose is present in the formulations at a concentration of about 40 to about 60 mg/ml.

iv. Buffering Agents, Buffering Systems, Ionic Strength, and pH

Buffering agents help to maintain the pH of the formulations in a range that approximates physiological conditions. Buffers, if present, are in the formulations at a concentration ranging from about 1 mM to about 50 mM. Suitable buffering agents for use with the instant invention include both organic and inorganic acids, and salts thereof, such as citrate buffers (e.g., monosodium citrate-disodium citrate mixture, citric acid-trisodium citrate mixture, citric acid-monosodium citrate mixture etc.), succinate buffers (e.g., succinic acid-monosodium succinate mixture, succinic acid-sodium hydroxide mixture, succinic acid-disodium succinate mixture etc.), tartrate buffers (e.g., tartaric acid-sodium tartrate mixture, tartaric acid-potassium tartrate mixture, tartaric acid-sodium hydroxide mixture etc.), fumarate buffers (e.g., fumaric acid-monosodium fumarate mixture, fumaric acid-disodium fumarate mixture, monosodium fumarate-disodium fumarate mixture etc.), gluconate buffers (e.g., gluconic acid-sodium glyconate mixture, gluconic acid-sodium hydroxide mixture, gluconic acid-potassium gluconate mixture etc.), oxalate buffers (e.g., oxalic acid-sodium oxalate mixture, oxalic acid-sodium hydroxide mixture, oxalic acid-potassium oxalate mixture etc.), lactate buffers (e.g., lactic acid-sodium lactate mixture, lactic acid-sodium hydroxide mixture, lactic acid-potassium lactate mixture etc.) and acetate buffers (e.g., acetic acid-sodium acetate mixture, acetic acid-sodium hydroxide mixture etc.). Phosphate buffers, carbonate buffers, histidine buffers, trimethylamine salts such as Tris, HEPES and other such known buffers are also suitable and can be used. In some embodiments, a combination of buffers, i.e., two or more buffering agents, is used in the formulations of the present invention. A combination of two or more buffers is referred to herein as a buffering system.

The formulations of the invention may, optionally, comprise a buffering system, as some embodiments of the invention do not contain any buffers. A buffering system maintains a physiologically suitable pH. In addition, a buffering system enhances isotonicity and chemical stability of the formulation. Due to the difficulty of developing a stable antibody formulation for the bispecific antibody, it is advantageous to use a buffering system in order to take advantage of the benefits of two or more buffers. By combining the benefits of two or more buffers, a more stable antibody formulation is able to be developed.

In some embodiments, the buffering system is present in the formulations at a concentration from about 1 mM to about 50 mM, e.g., about 5 mM to about 25 mM, about 5 mM to about 15 mM, about 8 mM, or about 10 mM. Alternatively, the buffering system is present in the formulations at a concentration from about 1 mM to about 15 mM, about 16 to about 30 mM, about 31 to about 45 mM, or about 46 mM to about 50 mM. For example, the buffering system may be present in the formulation at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, and about 50 mM. In some embodiments, the buffering system is present in the formulation at a concentration from about 5 mM to about 15 mM, and in some embodiments from about 8 mM to about 12 mM. In an embodiment, the buffering system is present at a concentration of about 10 mM. In another embodiment, the buffering system is present at a concentration of about 8 mM.

In some embodiments, the buffering system comprises a Tris buffer and a phosphate buffer. In some embodiments, the Tris buffer is present in the formulations at a concentration from about 1 to about 5 mM. For example, the Tris buffer may be present in the formulation at a concentration of about 1 mM, about 2 mM, about 3 mM, about 4 mM, or about 5 mM. In some embodiments, the Tris buffer is present in the formulations at a concentration from about 2 mM to about 4 mM, and in some embodiments from about 3 mM to about 4 mM. In an embodiment, the Tris buffer is present at a concentration of about 3.7 mM. In another embodiment, the Tris buffer is present at a concentration of about 3 mM.

In some embodiments, the phosphate buffer is present in the formulations at a concentration from about 1 to about 10 mM. For example, the phosphate buffer may be present in the formulations at a concentration of about 1 mM, about 2 mM, about 3 mM, about 4 mM, about 5 mM, about 6 mM, about 7 mM, about 8 mM, about 9 mM, or about 10 mM. In some embodiments, the phosphate buffer is present in the formulations at a concentration from about 3 mM to about 8 mM, and in some embodiments from about 5 mM to about 7 mM. In an embodiment, the phosphate buffer is present at a concentration of about 6.3 mM. In another embodiment, the phosphate buffer is present at a concentration of about 5 mM.

In an embodiment of the invention, the buffering system comprises a Tris buffer at a concentration of about 3.7 mM and a phosphate buffer at a concentration of about 6.3 mM. In another embodiment of the invention, the buffering system comprises a Tris buffer at a concentration of about 3 mM and a phosphate buffer at a concentration of about 5 mM. This combination of Tris buffer and phosphate buffer in a buffer system is highly unusual and is not known in the art.

In some embodiments, the buffering system is present in the formulations in a low concentration, i.e., about 15 mM or less, including 0 mM (no salt).

In certain embodiments, the formulations of the invention have a pH around pH 7. In some embodiments, the pH of the formulations range from about 5.0 to about 8.0. For example, the pH of the formulations may be about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, and about 8.0. In some embodiments, the pH of the formulations may range from about 6.5 to about 7.5. In an embodiment, the pH is about 7.0. The formulations exhibit good stability regarding high molecular weight proteins when the pH of the formulations is about pH 7. The pH of the formulation may be measured by any means known to those of skill in the art. A means for measuring pH is using a pH meter with a micro-electrode. The pH of the formulation may be adjusted using any means known in the art. Chemicals for altering the pH of the formulations are hydrochloric acid (HCl) and sodium hydroxide (NaOH).

In certain embodiments, the formulations of the invention have a pH above the isoelectric point (pI) of the antibody. The isoelectric point is the pH at which a particular molecule or surface carries no net electrical charge. The pI of the bispecific antibody may be determined by any means known to those of skill in the art. In some embodiments, the pI of the bispecific antibody is determined by denaturated isoelectric focusing. The pI of the anti-IL-4/anti-IL-13 bispecific antibody comprising a heavy chain variable region comprising the amino acid sequences of SEQ ID NOs: 2 and 4; and a light chain variable region comprising the amino acid sequences of SEQ ID NOs: 1 and 3 is 5.8-6.2.

v. Surfactants

The formulations of the invention may, optionally, further comprise a surfactant, as some embodiments of the invention do not contain any surfactants. Surfactants are chemical compounds that interact with and stabilize biological molecules or general pharmaceutical excipients in a formulation. Surfactants generally protect the molecules and excipients from air/solution interface induced stresses and solution/surface induced stresses, which may otherwise result in the aggregation of molecules. Surfactants also prevent visible and sub-visible particle formation.

In some embodiments, the surfactant is present in the formulations at a concentration from about 0.01% to about 0.5% (w/v), e.g., about 0.01% to about 0.3%, or about 0.01% to about 0.2%. Alternatively, the surfactant is present in the formulations at a concentration from about 0.01% to about 0.05% (w/v), about 0.06% to about 0.10% (w/v), about 0.11% to about 0.15% (w/v), about 0.16% to about 0.20% (w/v), about 0.20% to about 0.30% (w/v), about 0.30% to about 0.40% (w/v), or about 0.40% to about 0.50% (w/v). For example, the surfactant may be present in the formulations in an amount of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), and about 0.5% (w/v). In particular embodiments, the surfactant is present in the formulations from about 0.03% to about 0.2% (w/v).

Examples of surfactants include, but are not limited to, polysorbates, glycerin, dicarboxylic acids, oxalic acid, succinic acid, fumaric acids, phthalic acids, and combinations thereof. Those skilled in the art are aware that other surfactants, e.g. non-ionic or ionic detergents, can be used as long as they are pharmaceutically acceptable, i.e. suitable for administration to subjects. In some embodiments, the surfactant is a polysorbate. Examples of polysorbates include polysorbate 20, polysorbate 40, polysorbate 60, polysorbate 65, and polysorbate 80. In specific embodiments, the surfactant is polysorbate 80.

In exemplary embodiments, polysorbate 80 is present in the formulations in an amount from about 0.01% to about 1% (w/v). For example, polysorbate 80 may be present in the formulations in an amount of about 0.01% (w/v), about 0.02% (w/v), about 0.03% (w/v), about 0.04% (w/v), about 0.05% (w/v), about 0.06% (w/v), about 0.07% (w/v), about 0.08% (w/v), about 0.09% (w/v), about 0.1% (w/v), about 0.2% (w/v), about 0.3% (w/v), about 0.4% (w/v), about 0.5% (w/v), about 0.6% (w/v), about 0.7% (w/v), about 0.8% (w/v), about 0.9% (w/v), and about 1% (w/v). In particular embodiments, polysorbate 80 is present in the formulations from about 0.03% to about 0.2% (w/v). For example, polysorbate 80 may be present in an amount from about 0.01% to about 1% (w/v), about 0.02% to about 0.5% (w/v), and about 0.03% to about 0.2% (w/v). In some embodiments of the invention, polysorbate 80 is present in the formulations in an amount of 0.2% (w/v).

vi. Stabilizing Agents

The formulations of the invention may, optionally, further comprise a stabilizing agent, as some embodiments of the invention do not contain any stabilizing agents. Stabilizing agents refer to a broad category of excipients that can range in function from a bulking agent to an additive that solubilizes the therapeutic agent or helps to prevent denaturation or adherence to the container wall. Stabilizing agents also minimize high molecular weight protein formation.

In some embodiments, the stabilizing agent is present in the formulations at a concentration from about 1% to about 10% (w/v), e.g., about 2% to about 8% (w/v), about 2% to about 5% (w/v), about 2% to about 4% (w/v), or about 3% (w/v). Alternatively, the stabilizing agent is present in the formulations at a concentration from about 1% to about 2% (w/v), about 2% to about 4% (w/v), about 4% to about 6% (w/v), about 6% to about 8% (w/v), or about 8% to about 10% (w/v). For example, the stabilizing agent may be present in the formulations in an amount of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In particular embodiments, the stabilizing agent is present in the formulations from about 1% to about 5% (w/v), or from about 1% to about 3% (w/v), or about 3% (w/v).

Examples of stabilizing agents include, but are not limited to, polyhydric sugar alcohols; amino acids, such as proline, arginine, lysine, glycine, glutamine, asparagine, histidine, alanine, ornithine, L-leucine, 2-phenylalanine, glutamic acid, threonine etc.; organic sugars or sugar alcohols, such as lactose, trehalose, stachyose, arabitol, erythritol, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thiosulfate; low molecular weight polypeptides (i.e., <10 residues); proteins, such as human serum albumin, bovine serum albumin, gelatin or immunoglobulins; hydrophilic polymers, such as polyvinylpyrrolidone, saccharides, monosaccharides, such as xylose, mannose, fructose, glucose; disaccharides, such as lactose, maltose and sucrose; trisaccharides such as raffinose; polysaccharides such as dextran and so on. Those skilled in the art are aware that other stabilizing agents can be used as long as they are pharmaceutically acceptable, i.e. suitable for administration to subjects. In some embodiments, the stabilizing agent is an amino acid. In some embodiments, the stabilizing agent is proline or glycine. In some embodiments, the stabilizing agent is proline. Alternatively, the stabilizing agent is mannitol.

In certain embodiments, proline is present in the formulations in an amount from about 1% to about 10% (w/v). For example, proline may be present in the formulation in an amount of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, proline may be present in an amount of about 1% to about 5% (w/v), or about 1% to about 3% (w/v). In specific embodiments, proline is present in the formulations in an amount of about 3% (w/v).

In certain alternative embodiments, mannitol is present in the formulations in an amount from about 1% to about 10% (w/v). For example, mannitol may be present in the formulation in an amount of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, mannitol may be present in an amount of about 1% to about 5% (w/v), or about 1% to about 3% (w/v). In specific embodiments, mannitol is present in the formulations in an amount of about 3% (w/v).

vii. Other Excipients

Furthermore, the formulations of the invention may, optionally, further comprise other excipients including, but not limited to, water for injection, diluents, solubilizing agents, soothing agents, additional buffers, inorganic or organic salts, surfactants, stabilizing agents, amino acids, sugars, antioxidants, preservatives, bulking agents, chelating agents, tonicity agents, or the like. In some embodiments, however, the formulations of the invention comprise no other excipients, except those described above. Other pharmaceutically acceptable carriers, excipients, or stabilizers, such as those described in Remington's Pharmaceutical Sciences 16^{th} edition, Osol, A. Ed. (1980) may be included in the formulation provided that they do not adversely affect the desired characteristics of the formulation. In a particular embodiment, the formulation is substantially free of preservatives, although, in alternative embodiments, preservatives may be added as necessary. For example, cryoprotectants or lyoprotectants may be included in lyophilized formulations.

viii. Liquid or Lyophilized Formulations

The formulations of the invention may either be liquid formulations or lyophilized formulations. In some embodiments, the formulations are liquid formulations. In some embodiments, the liquid formulations are ready for injection. Alternatively, the formulations may be lyophilized powders. In some embodiments, the lyophilized powders are ready to be combined with a solvent just prior to administration.

ix. Exemplary Formulations

In an exemplary embodiment of the invention, the invention provides a stable liquid antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 10 mg/mL of a polyglutamate polymer randomly grafted with Vitamin E, wherein the polyglutamate polymer has a nominal degree of polymerization of 100, and 10% mol/mol of Vitamin E grafted to the polymer;

about 50 mg/g of sucrose; and

about 10 mM of salts composed of about 6.3 mM of phosphate buffer and about 3.7 mM of Tris buffer.

In one exemplary embodiment of the invention, the invention provides a stable liquid antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 10 mg/mL of a polyglutamate polymer randomly grafted with Vitamin E, wherein the polyglutamate polymer has a nominal degree of polymerization of 100, and 10% mol/mol of Vitamin E grafted to the polymer;

about 90 mg/g of sucrose; and

wherein this formulation contains no added salt.

In one exemplary embodiment of the invention, the invention provides a stable liquid antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 5 mg/mL of a polyglutamate polymer randomly grafted with Vitamin E, wherein the polyglutamate polymer has a nominal degree of polymerization of 50, and 10% mol/mol of Vitamin E grafted to the polymer;

about 50 mg/g of sucrose; and

about 10 mM of salts composed of about 6.3 mM of phosphate buffer and about 3.7 mM of Tris buffer.

In one exemplary embodiment of the invention, the invention provides a stable liquid antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 5 mg/mL of a polyglutamate polymer randomly grafted with Vitamin E, wherein the polyglutamate polymer has a nominal degree of polymerization of 50, and 10% mol/mol of Vitamin E grafted to the polymer;

about 90 mg/g of sucrose; and

wherein this formulation contains no added salt.

In one exemplary embodiment of the invention, the invention provides a stable liquid antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 10 mg/mL of a polyglutamate polymer randomly grafted with Vitamin E, wherein the polyglutamate polymer has a nominal degree of polymerization of 100, and 10% mol/mol of Vitamin E grafted to the polymer;

about 10 mM of a buffering system, wherein the buffering system comprises a Tris buffer concentration of about 3.7 mM and a Phosphate buffer concentration of about 6.3 mM;

about 0.2% (w/v) polysorbate 80;

about 5% (w/v) sucrose;

about 3% (w/v) proline; and

wherein the pH of the formulation is about pH 7.

In one exemplary embodiment of the invention, the invention provides a stable liquid antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 5 mg/mL of a polyglutamate polymer randomly grafted with Vitamin E, wherein the polyglutamate polymer has a nominal degree of polymerization of 50, and 10% mol/mol of Vitamin E grafted to the polymer;

about 10 mM of a buffering system, wherein the buffering system comprises a Tris buffer concentration of about 3.7 mM and a Phosphate buffer concentration of about 6.3 mM;

about 0.2% (w/v) polysorbate 80;

about 5% (w/v) sucrose;

about 3% (w/v) proline; and

wherein the pH of the formulation is about pH 7.

In another exemplary embodiment of the invention, the invention provides a stable liquid antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 10 mg/mL of a polyglutamate polymer randomly grafted with Vitamin E, wherein the polyglutamate polymer has a nominal degree of polymerization of 100, and 10% mol/mol of Vitamin E grafted to the polymer;

about 10 mM of a buffering system, wherein the buffering system comprises a Tris buffer concentration of about 3.7 mM and a phosphate buffer concentration of about 6.3 mM;

about 0.2% (w/v) polysorbate 80;

about 5% (w/v) sucrose;

about 3% (w/v) mannitol; and

wherein the pH of the formulation is about pH 7.

In an alternative exemplary embodiment of the invention, the invention provides a stable lyophilized antibody formulation suitable for subcutaneous administration, the formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 10 mg/mL of a polyglutamate polymer randomly grafted with Vitamin E, wherein the polyglutamate polymer has a nominal degree of polymerization of 100, and 10% mol/mol of Vitamin E grafted to the polymer;

about 10 mM of a buffering system, wherein the buffering system comprises a Tris buffer concentration of about 3.7 mM and a Phosphate buffer concentration of about 6.3 mM;

about 0.2% (w/v) polysorbate 80;

about 5% (w/v) sucrose;

about 3% (w/v) proline; and

wherein the pH of the formulation is about pH 7.

In an alternative exemplary embodiment of the invention, the invention provides a stable lyophilized antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 5 mg/mL of a polyglutamate polymer randomly grafted with Vitamin E, wherein the polyglutamate polymer has a nominal degree of polymerization of 50, and 10% mol/mol of Vitamin E grafted to the polymer;

about 10 mM of a buffering system, wherein the buffering system comprises a Tris buffer concentration of about 3.7 mM and a Phosphate buffer concentration of about 6.3 mM;

about 0.2% (w/v) polysorbate 80;

about 5% (w/v) sucrose;

about 3% (w/v) proline; and

wherein the pH of the formulation is about pH 7.

In another alternative exemplary embodiment of the invention, the invention provides a stable lyophilized antibody formulation suitable for subcutaneous administration, the formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 10 mg/mL of a polyglutamate polymer randomly grafted with Vitamin E, wherein the polyglutamate polymer has a nominal degree of polymerization of 100, and 10% mol/mol of Vitamin E grafted to the polymer;

about 10 mM of a buffering system, wherein the buffering system comprises a Tris buffer concentration of about 3.7 mM and a Phosphate buffer concentration of about 6.3 mM;

about 0.2% (w/v) polysorbate 80;

about 5% (w/v) sucrose;

about 3% (w/v) mannitol; and

wherein the pH of the formulation is about pH 7.

In another alternative exemplary embodiment of the invention, the invention provides a stable lyophilized antibody formulation comprising:

about 100 mg/mL of a bispecific antibody or an antigen binding fragment thereof, wherein the antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;

about 5 mg/mL of a polyglutamate polymer randomly grafted with Vitamin E, wherein the polyglutamate polymer has a nominal degree of polymerization of 50, and 10% mol/mol of Vitamin E grafted to the polymer;

about 10 mM of a buffering system, wherein the buffering system comprises a Tris buffer concentration of about 3.7 mM and a Phosphate buffer concentration of about 6.3 mM;

about 0.2% (w/v) polysorbate 80;

about 5% (w/v) sucrose;

about 3% (w/v) mannitol; and

wherein the pH of the formulation is about pH 7.

x. Stability

The formulations of the invention are stable at 2-8° C. for at least about 1, 2, 3, 4, 5, 6, 7, 8, 9, 10, 11, 12, 13, 14, 15, 16, 17, 18, 19, 20, 21, 22, 23, or 24 months or more. In exemplary embodiments, they are stable at 2-8° C. for at least about 6 months or more. In other exemplary embodiments, they are stable at 2-8° C. for at least about 9 months. In further exemplary embodiments, they are stable at 2-8° C. for at least about 1 year or more, such as about 2 years.

The formulations of the invention are stable at room temperature for at least about 24 hours.

### C. Modes of Administration

In certain embodiments of the invention, the formulations are suitable for administration parenterally, intravenously, intramuscularly, intradermally, subcutaneously, or a combination thereof. The formulations of the invention are suitable for delivery by a variety of techniques. In some embodiments of the invention, the formulation is administered subcutaneously. For example, some formulations containing 100 mg/mL of anti-IL-4/anti-IL-13 bispecific antibody are administered subcutaneously. Therefore, the formulations are sterile. Methods for making formulations sterile are well known in the art and include, for example, filtration through sterile filtration membranes or autoclaving the ingredients of the formulation, with the exception of the antibodies, at about 120° C. for about 30 minutes.

### D. Dosages and Dosage Forms

Effective doses of the formulations of the invention vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages may need to be titrated to optimize safety and efficacy. In some embodiments, the dose ranges from 100-200 mg/vial.

The formulations of the invention may be administered on multiple occasions. Intervals between single dosages can be daily, weekly, biweekly, monthly or yearly. Intervals can also be irregular. In some methods, the dosage is adjusted to achieve a certain plasma binding agent, such as an antibody, concentration. Dosage and frequency will vary depending on the half-life of the anti-IL-4/anti-IL-13 bispecific antibody in the subject. In general, human antibodies show the longest half-life, followed by humanized antibodies, chimeric antibodies, and nonhuman antibodies.

In further embodiments, the invention provides a pharmaceutical unit dosage form comprising a therapeutically effective amount of a formulation of the invention for the treatment of one or more diseases in a subject through administration of the dosage form to the subject. In an embodiment, the subject is a human. The human may be an adult or may be an infant. The term “pharmaceutical unit dosage form” refers to a physically discrete unit suitable as unitary dosages for the subjects to be treated, each unit containing a predetermined quantity of active compound calculated to produce the desired therapeutic/prophylactic effect in association with the required buffer and pH.

The unit dosage form may be a container comprising the formulation. Suitable containers include, but are not limited to, sealed ampoules, vials, bottles, syringes, and test tubes. The containers may be formed from a variety of materials, such as glass or plastic, and may have a sterile access port (for example, the container may be a vial having a stopper pierceable by a hypodermic injection needle). In an embodiment the container is a vial. Generally, the container should maintain the sterility and stability of the formulation.

In specific embodiments, the formulations are packaged in 7 or 15 mL vials that are made of clear, colorless type I glass, and closed with a stopper (fluoropolymer-coated bromobutyl) sealed with flip-of caps with flange (polypropylene).

In specific embodiment, the formulations are secondarily packaged in a container, such as a cardboard box, that protects the vials from light.

The formulations to be used for in vivo administration must be sterile. That can be accomplished, for example, by filtration through sterile filtration membranes. For example, the liquid formulations of the present invention may be sterilized by filtration using a 0.2 μm or a 0.22 μm filter.

### E. Methods of Treatment

Further provided herein are methods for treating an IL-4 or IL-13 or both-mediated disease or disorder, the methods comprising administering a formulation of the invention to a subject. In certain embodiments, the IL-4 or IL-13 or both-mediated disease is cancers, inflammation, autoimmune diseases, infections, cardiovascular diseases, respiratory diseases, neurological diseases and metabolic diseases.

The formulations of the present invention may be used to treat, suppress or prevent disease, such as an allergic disease, a Th2-mediated disease, IL-13-mediated disease, IL-4-mediated disease, or IL-4/IL-13-mediated disease. Examples of such diseases include, Hodgkin's disease, asthma, allergic asthma, atopic dermatitis, atopic allergy, ulcerative colitis, scleroderma, allergic rhinitis, COPD, idiopathic pulmonary fibrosis, chronic graft rejection, bleomycin-induced pulmonary fibrosis, radiation-induced pulmonary fibrosis, pulmonary granuloma, progressive systemic sclerosis, schistosomiasis, hepatic fibrosis, renal cancer, Burkitt lymphoma, Hodgkins disease, non-Hodgkins disease, Sezary syndrome, asthma, septic arthritis, dermatitis herpetiformis, chronic idiopathic urticaria, ulcerative colitis, scleroderma, hypertrophic scarring, Whipple's Disease, benign prostate hyperplasia, a lung disorder in which IL-4 receptor plays a role, condition in which IL-4 receptor-mediated epithelial barrier disruption plays a role, a disorder of the digestive system in which IL-4 receptor plays a role, an allergic reaction to a medication, Kawasaki disease, sickle cell disease, Churg-Strauss syndrome, Grave's disease, pre-eclampsia, Sjogren's syndrome, autoimmune lymphoproliferative syndrome, autoimmune hemolytic anemia, Barrett's esophagus, autoimmune uveitis, tuberculosis, cystic fibrosis, allergic bronchopulmonary mycosis, chronic obstructive pulmonary disease, bleomycin-induced pneumopathy and fibrosis, pulmonary alveolar proteinosis, adull respiratory distress syndrome, sarcoidosis, hyper IgE syndrome, idiopathic hypereosinophil syndrome, an autoimmune blistering disease, pemphigus vulgaris, bullous pemphigoid, myasthenia gravis, chronic fatigue syndrome, nephrosis.

The term “allergic disease” refers to a pathological condition in which a patient is hypersensitized to and mounts an immunologic reaction against a substance that is normally nonimmunogenic. Allergic disease is generally characterized by activation of mast cells by IgE resulting in an inflammatory response (e.g., local response, systemic response) that can result in symptoms as benign as a runny nose, to life-threatening anaphylactic shock and death. Examples of allergic disease include, but are not limited to, allergic rhinitis (e.g., hay fever), asthma (e.g., allergic asthma), allergic dermatitis (e.g., eczema), contact dermatitis, food allergy and urticaria (hives).

The term “Th2-mediated disease” refers to a disease in which pathology is produced (in whole or in part) by an immune response (Th2-type immune response) that is regulated by CD4⁺ Th2 T lymphocytes, which characteristically produce IL-4, IL-5, IL-9 and IL-13. A Th2-type immune response is associated with the production of certain cytokines (e.g., IL-4, IL-13) and of certain classes of antibodies (e.g., IgE), and is associate with humoral immunity. Th2-mediated diseases are characterized by the presence of elevated levels of Th2 cytokines (e.g., IL-4, IL-13) or certain classes of antibodies (e.g., IgE) and include, for example, allergic disease (e.g., allergic rhinitis, atopic dermatitis, asthma (e.g., atopic asthma), allergic airways disease (AAD), anaphylactic shock, conjunctivitis), autoimmune disorders associated with elevated levels of IL-4 or IL-13 or both (e.g., rheumatoid arthritis, host-versus-graft disease, renal disease (e.g., nephritic syndrome, lupus nephritis)), and infections associated with elevated levels of IL-4 or IL-13 or both (e.g., viral, parasitic, fungal (e.g., C. albicans) infection). Certain cancers are associated with elevated levels of IL-4 or IL-13 or both or associated with IL-4-induced or IL-13-induced or both-induced cancer cell proliferation (e.g., B cell lymphoma, T cell lymphoma, multiple myeloma, head and neck cancer, breast cancer and ovarian cancer). These cancers can be treated, suppressed or prevented using the formulations of the invention.

The term “cancer” refers to or describes the physiological condition in mammals, in particular humans, which is typically characterized by unregulated cell growth. Examples of cancer include, but are not limited to, carcinoma, lymphoma, blastoma, sarcoma, and leukemia.

The term “autoimmune disease” refers to a non-malignant disease or disorder arising from and directed against an individual's own tissues. Examples of autoimmune diseases or disorders include, but are not limited to, inflammatory responses such as inflammatory skin diseases including psoriasis and dermatitis; allergic conditions such as eczema and asthma; other conditions involving infiltration of T cells and chronic inflammatory responses; atherosclerosis; diabetes mellitus (e.g. Type I diabetes mellitus or insulin dependent diabetes mellitis); multiple sclerosis and central nervous system (CNS) inflammatory disorder.

In certain embodiments, the formulations of the invention can be administered in combination with one or more therapies (e.g., therapies that are not the formulations of the invention that are currently administered to prevent, treat, manage, or ameliorate an IL-4 or IL-13 or both-mediated disease. The use of the term “in combination” does not restrict the order in which therapies are administered to a subject. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject that had, has, or is susceptible to an IL-4 or IL-13 or both-mediated disease. Any additional therapy can be administered in any order with the other additional therapies. Non-limiting examples of therapies that can be administered in combination with an antibody of the invention include approved anti-inflammatory agents listed in the U.S. Pharmacopoeia or Physician's Desk Reference.

Certain embodiments of the invention include the use of a formulation described herein for the manufacture of a medicament for treating, suppressing, or preventing a disease or disorder described herein.

In other embodiments, the invention includes a composition comprising a formulation described herein for the treatment, suppression, or prevention of a disease or disorder described herein.

### F. Kits

Certain embodiments of the invention include a kit comprising a formulation of the invention. The kit may further comprise one or more containers comprising pharmaceutically acceptable excipients, and include other materials desirable from a commercial and user standpoint, including filters, needles and syringes. Associated with the kits can be instructions customarily included in commercial packages of therapeutic, prophylactic or diagnostic products, that contain information about, for example, the indications, usage, dosage, manufacture, administration, contra-indications, or warnings concerning the use of such therapeutic, prophylactic or diagnostic products.

### EXAMPLES

To help illustrate the invention, the following examples are provided. The examples are not intended to limit the scope of the invention in any way. In general, the practice of the present invention employs, unless otherwise indicated, conventional techniques of pharmaceutical formulation, chemistry, molecular biology, recombinant DNA technology, immunology such as antibody technology, and standard techniques of polypeptide preparation as described, for example, in Sambrook, Fritsch and Maniatis, Molecular Cloning: Cold Spring Harbor Laboratory Press (1989); Antibody Engineering Protocols (Methods in Molecular Biology), volume 51, Ed.: Paul S., Humana Press (1996); Antibody Engineering: A Practical Approach (Practical Approach Series, 169), Eds.: McCafferty J. et al., Humana Press (1996); Antibodies: A Laboratory Manual, Harlow and Lane, Cold Spring Harbor Laboratory Press (1999); and Current Protocols in Molecular Biology, Eds. Ausubel et al., John Wiley & Sons (1992).

A humanized IgG4 anti-IL-4/anti-IL-13 bispecific antibody comprising a heavy chain variable region that binds to both IL-13 and IL-4 comprising the amino acid sequences of SEQ ID NOs: 2 and 4, and a light chain variable region that binds to both IL-13 and IL-4 comprising the amino acid sequences of SEQ ID NOs: 1 and 3 (the “Antibody”) was used in the following examples in order to determine optimal formulation conditions. Additional details regarding the exemplary humanized IgG4 anti-IL-4/anti-IL-13 bispecific antibody can be found in U.S. Pat. No. 8,388,965, which is incorporated herein by reference in its entirety.

In these examples, the number of glutamic acid units in the PGA polymer chain, i.e., the nominal degree of polymerization, has been varied as well as the mol/mol of Vitamin E grafted to the polymer. The below table summarizes the different PGA polymer structures used in the examples.

| Polymer | Degree of | Grafting level expressed |
|---|---|---|
| nomenclature | polymerization | in molar % of Vitamin E |
| P1 | 100 | 5 |
| P2 | 100 | 10 |
| P3 | 100 | 15 |
| P4 | 50 | 5 |
| P5 | 50 | 10 |
| P6 | 220 | 10 |

The concentration of the Antibody was 100 mg/mL, and two different molar ratios of Antibody versus the PGA polymer were tested (1/1 and 1/3).

### SEC Method

The determination of the High Molecular Weight content, reported as area percentage in the formulations, was done using Size Exclusion Chromatography (SEC), with fluorescence detection. It was necessary to dissociate the Antibody from the polymer before SEC analysis, without dissociating the Antibody aggregates or High Molecular Weight species (HMW). This was done by diluting the sample up to 2.5 g/mL with a 16 mM sodium phosphate buffer pH 7.3 containing 5% (w/v) of Tween 20. 0.1% (w/v) of Tween 20 was further added in the mobile phase.

The analytical conditions as well as the sample preparation are summarized in the table below.

| | |
|---|---|
| Columns | Two ProSEC300S (250 × 4.6 mm) in series |
| Mobile phase | 0.1M sodium phosphate/0.2M sodium chloride/0.1% |
| | (w/v) Tween 20, pH 7.0 |
| Flow rate | 0.2 mL/min |
| Colum temperature | 35° C. |
| Auto sampler | 5° C. |
| temperature | |
| Detection | FLD (Exc: 280 nm; Em: 343 nm) |
| Run time | 40 min |
| Sample preparation | The sample is diluted up to 2.5 g/mL with a 16 mM |
| | sodium phosphate buffer pH 7.3 containing 5% |
| | (w/v) of Tween 20. |
| Injected volume | 10 μL |

### Example 1

### Preparation of a Ready-to-Use a Solution of Polymer P1 and the Antibody in a 1:1 Molar Ratio

### Stage 1: Preparation of the P1 Solution

0.106 g of water for injection was added to 0.030 g of a solution of polymer P1 at 45.03 mg/g: the P1 polymer concentration was 9.99 mg/g. The solution was maintained under moderate stirring (15 rpm) for 15 minutes at room temperature.

### Stage 2: Preparation of the Ready-to-Use Liquid Formulation

The Antibody was available as a lyophilisate form, hereafter called the Drug Product, with the following composition:
Antibody: 100 mg/ml
Phosphate Buffer: 6.34 mM
Tris Buffer pH7.2: 3.66 mM
Sucrose: 5% w/v
Proline: 3% w/v
Tween 80: 0.2% w/v

27.8 mg of the Drug Product was weighed in a vial. 0.136 g of the prepared P1 solution was slowly added onto the Drug Product. The vial was stirred for 10 minutes at room temperature on a roller stirrer. The obtained clear liquid contained 90.51 mg of Antibody and 8.30 mg of polymer P1 per mL.

### Stage 3: Analysis of the Antibody/P1 Polymer Liquid Formulation

After 24 hours at room temperature, the Antibody recovery (Measured Antibody concentration×100/Theoretical Antibody concentration) was measured by SEC, as well as the content of HMW. The aggregation rate (% HMW/h) (defined as

$$\frac{\% {HMW}_{TXh} - \% {HMW}_{T 0}}{Xh} )$$

was calculated. As a comparison, the Antibody alone was also studied in the same conditions. The results are reported in the table below.

| Formulation | Assays | T0 | 24 h at RT |
|---|---|---|---|
| Antibody/Pi | Protein recovery (%) | 81 | 96 |
| 1:1 molar ratio | HMW (%) | 4.8 | 10.3 |
| | Aggregation rate (% HMW/h) | — | 0.2 |
| Antibody alone | Protein recovery (%) | 91 | 97 |
| | HMW (%) | 4.0 | 13.1 |
| | Aggregation rate (% HMW/h) | — | 0.4 |

The use of the P1 polymer at a 1:1 molar ratio with the Antibody prevented the Antibody from aggregation over a period of time of 24 hours.

### Example 2

### Preparation of a Ready-to-Use Solution of Polymer P2 and the Antibody in a 1:1 Molar Ratio

A 10.95 mg/g P2 polymer solution was prepared by diluting with water a primary solution containing 49.6 mg/g of polymer P2. 0.143 g of the prepared P2 solution was added using the same protocol as described in Example 1 to 29.1 mg of the Drug Product. The obtained clear solution contained 90.44 mg of Antibody and 9.10 mg of polymer P2 per mL.

The analytical results are reported in the table below.

| Formulation | Assays | T0 | 24 h at RT |
|---|---|---|---|
| Antibody/P2 | Protein recovery (%) | 92 | 95 |
| 1:1 molar ratio | HMW (%) | 4.3 | 8.3 |
| | Aggregation rate (% HMW/h) | — | 0.2 |
| Antibody alone | Protein recovery (%) | 91 | 97 |
| | HMW (%) | 4.0 | 13.1 |
| | Aggregation rate (% HMW/h) | — | 0.4 |

The use of the P2 polymer at a 1:1 molar ratio with the Antibody prevented the Antibody from aggregation.

### Example 3

### Preparation of a Ready-to-Use Solution of Polymer P3 and the Antibody at a 1:1 Molar Ratio

A 12.59 mg/g P3 polymer solution was prepared by diluting with water a primary solution containing 54.99 mg/g of polymer P3. 0.138 g of the prepared P3 solution was added using the protocol described in Example 1 to 27.9 mg of the Drug Product. The obtained clear solution contained 89.52 mg of Antibody and 10.48 mg of polymer P3 per mL.

The analytical results are reported in the table below.

| Formulation | Assays | T0 | 24 h at RT |
|---|---|---|---|
| Antibody/P3 | Protein recovery (%) | 105 | 121 |
| 1:1 molar ratio | HMW (%) | 4.1 | 15.8 |
| | Aggregation rate (% HMW/h) | — | 0.5 |
| Antibody alone | Protein recovery (%) | 91 | 97 |
| | HMW (%) | 4.0 | 13.1 |
| | Aggregation rate (% HMW/h) | — | 0.4 |

The use of the P3 polymer at a 1:1 molar ratio with the Antibody did not prevent the Antibody from aggregation.

### Example 4

### Preparation of a Ready-to-Use Solution of Polymer P4 and the Antibody at a 1:1 Molar Ratio

A 5.70 mg/g P4 polymer solution was prepared by diluting with water a primary solution containing 24.99 mg/g of polymer P4. 0.142 g of the prepared P4 solution was added using the protocol described in Example 1 to 28.3 mg of the Drug Product. The obtained clear solution contained 88.46 mg of Antibody and 4.76 mg of polymer P4 per mL.

The analytical results are reported in the table below.

| Formulation | Assays | T0 | 24 h at RT |
|---|---|---|---|
| Antibody/P4 | Protein recovery (%) | 96 | 91 |
| 1:1 molar ratio | HMW (%) | 4.7 | 9.3 |
| | Aggregation rate (% HMW/h) | — | 0.2 |
| Antibody alone | Protein recovery (%) | 91 | 97 |
| | HMW (%) | 4.0 | 13.1 |
| | Aggregation rate (% HMW/h) | — | 0.4 |

The use of the P4 polymer at a 1:1 molar ratio with the Antibody prevented the Antibody from aggregation over a period of time of 24 hours.

### Example 5

### Preparation of a ready-to-use solution of polymer P5 and the Antibody at a

1:1 molar ratio

A 5.59 mg/g P5 polymer solution was prepared by diluting with water a primary solution containing 25.05 mg/g of polymer P5. 0.171 g of the prepared P5 solution was added using the protocol described in Example 1 to 34.5 mg of the Drug Product. The obtained clear solution contained 89.67 mg of Antibody and 4.65 mg of polymer P5 per mL.

The analytical results are reported in the table below.

| Formulation | Assays | T0 | 24 h at RT |
|---|---|---|---|
| Antibody/P5 | Protein recovery (%) | 95 | 92 |
| 1:1 molar ratio | HMW (%) | 4.4 | 9.0 |
| | Aggregation rate (% HMW/h) | — | 0.2 |
| Antibody alone | Protein recovery (%) | 91 | 97 |
| | HMW (%) | 4.0 | 13.1 |
| | Aggregation rate (% HMW/h) | — | 0.4 |

The use of the P5 polymer at a 1:1 molar ratio with the Antibody prevented the Antibody from aggregation.

### Example 6

### Preparation of a Ready-to-Use Solution of Polymer P6 and the Antibody at a 1:1 Molar Ratio

A 26.12 mg/g P6 polymer solution was prepared by diluting with water a primary solution containing 61.5 mg/g of polymer P6. 0.258 g of the prepared P6 solution was added using the protocol described in Example 1 to 31.4 mg of the Drug Product. The obtained clear solution contained 99.05 mg of Antibody and 21.27 mg of polymer P6 per mL.

The analytical results are reported in the table below.

| Formulation | Assays | T0 | 24 h at RT |
|---|---|---|---|
| Antibody/P6 | Protein recovery (%) | 94 | 100 |
| 1:1 molar ratio | HMW (%) | 4.3 | 12.4 |
| | Aggregation rate (% HMW/h) | — | 0.3 |
| Antibody alone | Protein recovery (%) | 91 | 97 |
| | HMW (%) | 4.0 | 13.1 |
| | Aggregation rate (% HMW/h) | — | 0.4 |

The use of the P6 polymer at a 1:1 molar ratio with the Antibody did not prevent the Antibody from aggregation

### Example 7

### Preparation of a Ready-to-Use Solution of Polymer P2 and the Antibody in a 1:3 Molar Ratio

A 34.94 mg/g P2 polymer solution was prepared by diluting with water a primary solution containing 49.6 mg/g of P2 polymer. 0.137 g of the prepared P2 solution was added using the protocol described in Example 1 to 28.1 mg of the Drug Product. The obtained clear solution contained 90.82 mg of Antibody and 28.99 mg of polymer P2 per mL.

The analytical results are reported in the table below.

| Formulation | Assays | T0 | 24 h at RT |
|---|---|---|---|
| Antibody/P2 | Protein recovery (%) | 102 | 101 |
| 1:3 molar ratio | HMW (%) | 5.8 | 11.6 |
| | Aggregation rate (% HMW/h) | — | 0.2 |
| Antibody alone | Protein recovery (%) | 91 | 97 |
| | HMW (%) | 4.0 | 13.1 |
| | Aggregation rate (% HMW/h) | — | 0.4 |

The combination of the Antibody and the P2 polymer in a 1:3 molar ratio did not prevent the Antibody from aggregation.

### Example 8

### Preparation of a Ready-to-Use Solution of the Antibody and Polymer P5 in a 1:3 Molar Ratio

A 16.985 mg/g P5 polymer solution was prepared by diluting with water a primary solution containing 25.05 mg/g of polymer P5. 0.1565 g of the prepared P5 solution was added using the protocol described in Example 1 to 31.2 mg of the Drug Product. The obtained clear solution contained 88.70 mg of Antibody and 14.16 mg of polymer P5 per mL.

The analytical results are reported in the table below.

| Formulation | Assays | T0 | 24 h at RT |
|---|---|---|---|
| Antibody/P5 | Protein recovery (%) | 97 | 96 |
| 1:3 molar ratio | HMW (%) | 5.6 | 12.2 |
| | Aggregation rate (% HMW/h) | — | 0.3 |
| Antibody alone | Protein recovery (%) | 91 | 97 |
| | HMW (%) | 4.0 | 13.1 |
| | Aggregation rate (% HMW/h) | — | 0.4 |

The combination of the Antibody and the P5 polymer in a 1:3 molar ratio did not prevent the Antibody from aggregation.

### Example 9

### Preparation of a Freeze-Dried Combination of the Antibody and the Polymer P2 in a 1:1 Molar Ratio and Further Extemporaneous Reconstitution with Water for Injection

### Stage 1: Preparation of the P2 Solution

0.398 g of water for injection was added to a solution of 0.103 g of a primary solution containing 50.15 mg/g of polymer P2 at: the P2 polymer concentration was 10.28 mg/g. The solution was maintained under moderate stirring (15 rpm) for 10 minutes at room temperature.

### Stage 2: Preparation of the Antibody Solution

0.215 g of the Drug Product was weighed in a vial. 0.935 g of water for injection was added on the Drug Product and the vial was stirred for 10 minutes at room temperature on a roller stirrer. The Antibody concentration was 100.03 mg/g. The vial was kept 5 minutes at 4° C.

### Stage 3: Preparation of the Liquid Combination of Antibody and Polymer P2 in a 1:1 Molar Ratio

0.356 g of the prepared Antibody solution was slowly added onto 0.356 g of the prepared P2 solution. The vial was stirred for 5 minutes at 4° C. on a roller stirrer. The solution contained 50.04 mg/g of the Antibody and 5.13 mg/g of the polymer P2.

### Stage 4: Freeze-Drying of the Antibody/Polymer P2 Solution

The Antibody/Polymer P2 solution was lyophilized in a USIFROID freeze-dryer during 76 hours in order to obtain a solid composition.

### Stage 5: Reconstitution of the Antibody/Polymer P2 Ready-to-Use Solution

0.287 g of water for injection was slowly added onto the freeze-dried Antibody/Polymer P2 formulation. The vial was stirred for 10 minutes at 4° C. on a roller stirrer. The reconstituted clear solution contained 100.00 mg of Antibody and 10.26 mg of polymer P2 per mL.

### Stage 6: Analysis of the Reconstituted Antibody/Polymer P2 Solution

The analytical results are reported in the table below.

| Formulation | Assays | T0 | 24 h at RT |
|---|---|---|---|
| Antibody/P2 | Protein recovery (%) | 94 | 101 |
| 1:1 molar ratio | HMW (%) | 5.2 | 11.0 |
| | Aggregation rate (% HMW/h) | — | 0.2 |
| Antibody alone | Protein recovery (%) | 97 | 95 |
| (Freeze-dried in | HMW (%) | 4.3 | 13.7 |
| the same conditions) | Aggregation rate (% HMW/h) | — | 0.4 |

The combination of the Antibody and the P2 polymer in a 1:1 molar ratio prevented the Antibody from aggregation.

### Example 10

### Preparation of a Freeze-Dried Combination of the Antibody Drug Product and the Polymer P5 in a 1:1 Molar Ratio and Further Extemporaneous Reconstitution with Water for Injection

A 5.08 mg/g P5 polymeric solution was prepared by diluting with water a primary solution containing 25.05 mg/g of polymer P5. 0.365 g of a 100.03 mg/g Antibody Drug Product solution, prepared according the protocol described in step 2 of example 9, was added onto 0.354 g of the prepared P5 solution. The solution containing 50.78 mg/g of the Antibody and 2.50 mg/g of the Polymer P5 was freeze-dried.

The reconstituted ready-to-use solution was obtained by adding 0.2955 g of water for injection and 10 minute roller stirring at 4° C.

The reconstituted clear ready-to-use solution contained 99.92 mg of the Antibody and 4.92 mg of polymer P5 per mL.

The analytical results are reported in the table below.

| Formulation | Assays | T0 | 24 h at RT |
|---|---|---|---|
| Antibody/P5 | Protein recovery (%) | 94 | 102 |
| 1:1 molar ratio | HMW (%) | 4.8 | 10.1 |
| | Aggregation rate (% HMW/h) | — | 0.2 |
| Antibody alone | Protein recovery (%) | 97 | 95 |
| (Freeze-dried under | HMW (%) | 4.3 | 13.7 |
| the same conditions) | Aggregation rate (% HMW/h) | — | 0.4 |

The combination of the Antibody and the P5 polymer in a 1:1 molar ratio prevented the Antibody from aggregation.

### Example 11

### Preparation of a Freeze-Dried Combination of the Antibody Drug Product and the Polymer P2 in a 1:1 Molar Ratio and Further Extemporaneous Reconstitution with Water for Injection

### Stage 1: Preparation of the P2 Solution

0.544 g of water for injection was added to a solution of 0.060 g of polymer P2 at 50.15 mg/g: the P2 polymer concentration was 5.00 mg/g. The solution was maintained under moderate stirring (15 rpm) for 10 minutes at room temperature.

### Stage 2: Preparation of the Antibody Solution

0.1165 g of the Drug Product was weighed in a vial. 1.121 g of water for injection was added on the Drug Product. The vial was stirred for 10 minutes at room temperature on a roller stirrer. The Antibody concentration was 50.22 mg/g. The solution was kept 5 minutes at 4° C.

### Stage 3: Preparation of the Liquid Combination of the Antibody and the Polymer P2 in a 1:1 Molar Ratio

0.409 g of the prepared Antibody solution was slowly added onto 0.402 g of the prepared P2 solution. The vial was stirred for 5 minutes at 4° C. on a roller stirrer. The composition contained 25.30 mg/g of the Antibody and 2.48 mg/g of the Polymer.

### Stage 4: Freeze-Drying of the Antibody/Polymer P2 Liquid Combination

The liquid combination was lyophilized in a USIFROID freeze-dryer during 76 hours.

### Stage 5: Reconstitution of the Antibody/Polymer P2 Ready-to-Use Solution

0.165 g of water for injection was slowly added onto the Antibody/Polymer P2 freeze-dried combination. The vial was stirred for 10 minutes at 4° C. on a roller stirrer.

### Stage 6: Analysis of the Ready-to-Use Solution of Antibody and Polymer P2

The obtained clear solution contained 100.19 mg of Antibody and 9.81 mg of polymer P2 per mL.

The results are reported in the table below.

| Formulation | Assays | T0 | 24 h at RT |
|---|---|---|---|
| Antibody/P2 | Protein recovery (%) | 90 | 92 |
| 1:1 molar ratio | HMW (%) | 4.5 | 8.8 |
| | Aggregation rate (% HMW/h) | — | 0.2 |
| Antibody alone | Protein recovery (%) | 92 | 91 |
| (Freeze-dried in | HMW (%) | 4.1 | 14.1 |
| the same conditions) | Aggregation rate (% HMW/h) | — | 0.4 |

The combination of the Antibody and the polymer P2 in a 1:1 molar ratio prevented the Antibody from aggregation.

### Example 12

### Preparation of a Freeze-Dried Combination of the Antibody Drug Product and the Polymer P5 in a 1:1 Molar Ratio and Further Extemporaneous Reconstitution with Water for Injection

A 2.51 mg/g P5 polymer solution was prepared by diluting with water a primary solution containing 25.05 mg/g of polymer P5. 0.403 g of the Antibody Drug Product reconstituted solution at 50.22 mg/g (from step 2 of example 11) was added onto 0.402 g of the prepared P5 solution. The obtained solution containing 25.12 mg/g of the Antibody and 1.25 mg/g of the Polymer P5 was freeze-dried.

The reconstituted ready-to-use solution was obtained by adding 0.163 g of water for injection and 10 minutes roller stirring at 4° C.

The obtained clear solution contained 100.47 mg of Antibody and 5.01 mg of polymer P5 per mL.

The results are reported in the table below.

| Formulation | Assays | T0 | 24 h at RT |
|---|---|---|---|
| Antibody/P5 | Protein recovery (%) | 99 | 93 |
| 1:1 molar ratio | HMW (%) | 4.1 | 8.7 |
| | Aggregation rate (% HMW/h) | — | 0.2 |
| Antibody alone | Protein recovery (%) | 92 | 91 |
| (Freeze-dried in | HMW (%) | 4.1 | 14.1 |
| the same conditions) | Aggregation rate (% HMW/h) | — | 0.4 |

The change in the formulation process while freeze-drying the Antibody/P5 Polymer at a lower concentration did not lead to an improvement of the efficiency of the stabilization of the Antibody against aggregation, compared to example 5 where the Antibody Drug Product was directly reconstituted at 100 mg/ml with a P5 polymer solution at a 1/1 molar ratio. The combination of the Antibody and the polymer P5 in a 1:1 molar ratio prevented the Antibody from aggregation.

### Example 13

### Preparation of a Freeze-Dried Antibody in the Presence of Sucrose and Further Extemporaneous Reconstitution with Water for Injection as a Reference

An Antibody primary formulation with the following composition was used:
Antibody: 42 mg/ml
Sucrose: 2.1% w/v
Tween 80: 0.021% w/v

### Stage 1: Preparation of the Antibody Solution

A frozen solution of Antibody primary formulation was thawed at ambient temperature.

### Stage 2: Preparation of the Liquid Solution Antibody Containing Sucrose

0.580 g of the Antibody solution was slowly added onto 0.586 g of water for injection. The vial was stirred for 10 minutes at 4° C. on a roller stirrer. The Antibody concentration was 20.80 mg/g. 11.8 mg of sucrose was added and the vial was stirred for 10 minutes at 4° C. on a roller stirrer. The final sucrose was 20.95 mg/g.

### Stage 3: Freeze-Drying of the Antibody in the Presence of Sucrose

The solution prepared at stage 2 was lyophilized in a USIFROID freeze-dryer during 76 hours.

### Stage 4: Reconstitution of the Ready-to-Use Antibody Solution

0.181 g of water for injection was slowly added onto the lyophilized Antibody of stage 3. The vial was stirred for 10 minutes at 4° C. on a roller stirrer. The clear obtained solution contained 106.02 mg of Antibody and 107.42 mg of sucrose per ml.

The results are reported in the tables below.

| | Assays | T0 | 24 h at RT |
|---|---|---|---|
| Freeze-dried | Protein recovery (%) | 107 | 103 |
| Antibody alone | HMW (%) | 3.3 | 12.1 |
| | Aggregation rate (% HMW/h) | — | 0.4 |

### Example 14

### Preparation of a Freeze-Dried Combination of the Antibody and the Polymer P2 in a 1:1 Molar Ratio and Further Extemporaneous Reconstitution with Water for Injection

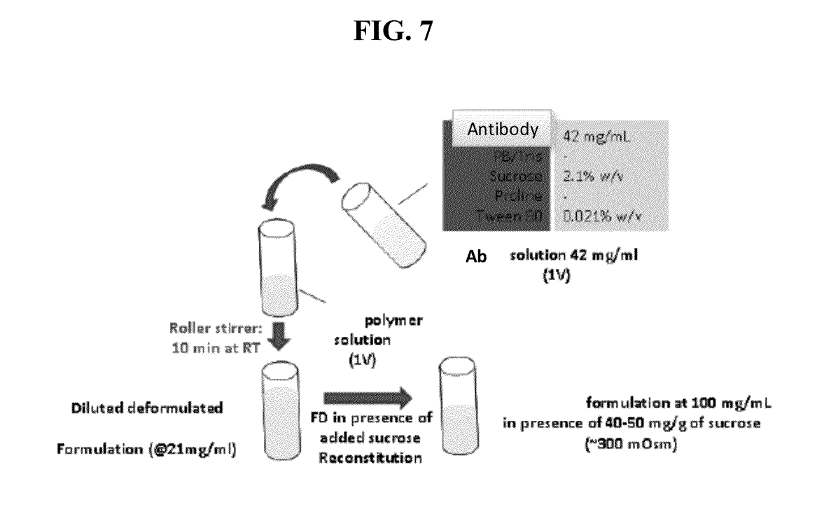

This protocol is similar to the one described in example 9 but stage 2 takes place with the Antibody Deformulated Substance (DS) described in example 15. See FIG. 7

### Stage 1: Preparation of the P2 Solution

1.3858 g of water for injection was added to 0.142 g of a primary solution of polymer P2 at 50.15 mg/g: the P2 polymer concentration was 3.90 mg/g. The solution was maintained under moderate stirring (15 rpm) for 5 minutes at room temperature.

### Stage 2: Preparation of the Antibody Solution

A frozen solution of the Antibody primary formulation of example 13 was thawed at ambient temperature.

### Stage 3: Preparation of the Liquid Combination of the Antibody and the Polymer P2 in a 1:1 Molar Ratio

0.577 g of the Antibody solution of stage 2 was slowly added onto 0.612 g of the prepared P2 solution. The vial was stirred for 10 minutes at 4° C. on a roller stirrer. The obtained solution contained 20.39 mg/g of the Antibody and 2.01 mg/g of the Polymer P2. 9.2 mg of sucrose was added and the vial was stirred for 10 minutes at 4° C. on a roller stirrer. The final sucrose concentration was 18.27 mg/g.

### Stage 4: Freeze-Drying of the Antibody/Polymer P2 Formulation in the Presence of Sucrose

The solution of stage 3 was lyophilized in a USIFROID freeze-dryer during 76 hours.

### Stage 5: Reconstitution of the Ready-to-Use Solution of Antibody/Polymer P2

0.181 g of water for injection was slowly added onto the Antibody/Polymer P2 freeze-dried combination. The vial was stirred for 10 minutes at 4° C. on a roller stirrer. The obtained clear solution contained 99.34 mg of Antibody, 9.77 mg of polymer P2, and 89.71 mg of sucrose per mL.

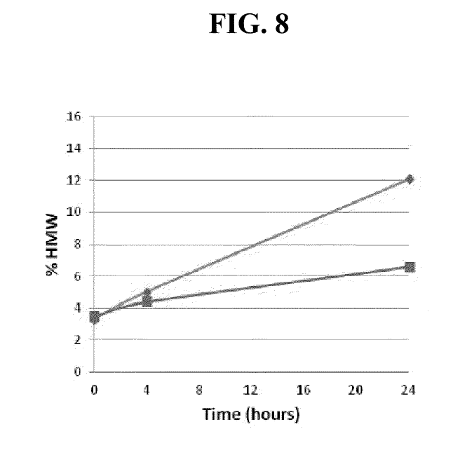

The results are reported in the tables below (see FIG. 8).

| Formulation | Assays | T0 | 24 h at RT |
|---|---|---|---|
| Antibody/P2 | Protein recovery (%) | 99 | 96 |
| 1:1 molar ratio | HMW (%) | 3.9 | 6.6 |
| | Aggregation rate (% HMW/h) | — | 0.1 |
| Antibody alone | Protein recovery (%) | 107 | 103 |
| (example 13) | HMW (%) | 3.3 | 12.1 |
| | Aggregation rate (% HMW/h) | — | 0.4 |

The combination of the P2 polymer and the Antibody in a 1:1 molar ratio prevented the Antibody from aggregation.

### Example 15

### Preparation of a Freeze-Dried Combination of Antibody and Polymer P5 in a 1:1 Molar Ratio and Further Extemporaneous Reconstitution with Water for Injection

A 1.94 mg/g P5 polymer solution was prepared by diluting with water a primary solution containing 25.05 mg/g of the polymer P5. 0.581 g of the Antibody solution at 42 mg/g from example 13 was added onto 0.5815 g of the prepared P5 solution, then 10.8 mg of sucrose was added. The obtained solution containing 20.79 mg/g of the Antibody, 0.96 mg/g of the Polymer P5, and 20.12 mg/g of sucrose was freeze-dried. The ready-to-use solution was obtained by adding 0.1815 g of water for injection to the freeze-dried combination and 10 minutes roller stirring at 4° C.

The obtained clear solution contained 99.55 mg of Antibody, 4.60 mg of polymer P5, and 96.34 mg of sucrose per mL.

The results are reported in the table below.

| Formulation | Assays | T0 | 24 h at RT |
|---|---|---|---|
| Antibody/P5 | Protein recovery (%) | 104 | 101 |
| 1:1 molar ratio | HMW (%) | 3.5 | 6.6 |
| | Aggregation rate (% HMW/h) | — | 0.1 |
| Antibody alone | Protein recovery (%) | 107 | 103 |
| (example 13) | HMW (%) | 3.3 | 12.1 |
| | Aggregation rate (% HMW/h) | — | 0.4 |

The combination of the P5 polymer and the Antibody in a 1:1 molar ratio prevented the Antibody from aggregation.

### Example 16

### Preparation of a Freeze-Dried Combination of the Antibody in the Presence of Sucrose and Further Extemporaneous Reconstitution with Water for Injection as a Reference

0.269 g of the Antibody primary solution of example 13 was diluted with water at 20.04 mg/g. 8.1 mg of sucrose was added. The final sucrose concentration was 24.54 mg/g. After freeze-drying, the clear reconstituted solution was obtained by adding 0.0805 g of water and contained 99.49 mg of Antibody and 123.62 mg of sucrose per mL.

The results are reported in the table below.

| | Assays | T0 | 24 h at RT |
|---|---|---|---|
| Freeze-dried | Protein recovery (%) | 100 | 100 |
| Antibody alone | HMW (%) | 3.9 | 15.1 |
| | Aggregation rate (% HMW/h) | — | 0.5 |

### Example 17

### Preparation of a Freeze-Dried Combination of the Antibody and the Polymer P5 in a 1:1 Molar Ratio and Further Extemporaneous Reconstitution with Water for Injection

A 1.95 mg/g P5 polymer solution was prepared by diluting with water a primary solution containing 25.05 mg/g of polymer P5. 0.268 g of the Antibody solution at 42 mg/g of example 13 was added onto 0.296 g of the prepared P5 solution, then 7.8 mg of sucrose was added. The obtained solution containing 19.66 mg/g of the Antibody, 1.01 mg/g of the Polymer P5, and 24.01 mg/g of sucrose was freeze-dried.

The ready-to-use solution was obtained by adding 0.081 g of water for injection and 10 minutes roller stirring at 4° C.

The clear obtained solution contained 98.28 mg of Antibody, 5.03 mg of polymer P5, and 119.99 mg of sucrose per mL.

The results are reported in the table below.

| Formulation | Assays | T0 | 24 h at RT |
|---|---|---|---|
| Antibody/P5 | Protein recovery (%) | 101 | 101 |
| 1:1 molar ratio | HMW (%) | 4.3 | 9.2 |
| | Aggregation rate (% HMW/h) | — | 0.2 |
| Antibody alone | Protein recovery (%) | 100 | 100 |
| (example 16) | HMW (%) | 3.9 | 15.1 |
| | Aggregation rate (% HMW/h) | — | 0.5 |

The combination of the P5 polymer and the Antibody in a 1:1 molar ratio prevented the Antibody from aggregation.

### Example 18

### Preparation of a Freeze-Dried Formulation of Antibody in the Presence of Sucrose and Further Extemporaneous Reconstitution with Water for Injection as a Reference

Example 13 was reproduced by another entity (B) with the same protocol.

### Stage 1: Preparation of the Antibody Solution

A frozen solution of Antibody at 42 mg/ml of example 13 was thawed at ambient temperature.

### Stage 2: Preparation of the Liquid Solution of Antibody Containing Sucrose

6.736 g of the Antibody solution was slowly added onto 7.290 g of water for injection. The flask was stirred for 10 minutes at 4° C. on a roller stirrer. The Antibody concentration was 20.17 mg/g. 117.2 mg of sucrose was added and the flask was stirred for 10 minutes at 4° C. on a roller stirrer. The final sucrose concentration was 18.29 mg/g.

### Stage 3: Freeze-Drying of the Antibody Solution Containing Sucrose

The solution was distributed within 10 vials containing approximately 1.2 ml. The vials were then lyophilized during 76 hours.

### Stage 4: Reconstitution of the Antibody Solution

Approximately 0.197 g of water for injection was slowly added into each lyophilized Antibody vial. Each vial was stirred for 10 minutes at 4° C. on a roller stirrer. The obtained clear solution contained approximately 100.0 mg of Antibody and 91.4 mg of sucrose per mL.

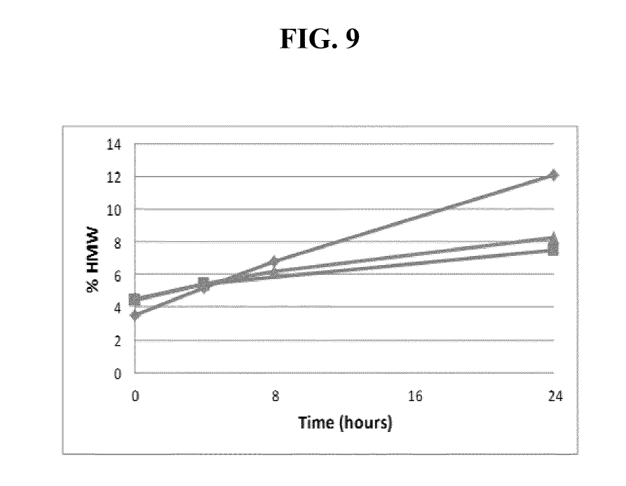

The results are reported in the table below (see FIG. 9).

| | Assays | T0 | 24 h at RT |
|---|---|---|---|
| Freeze-dried | Protein recovery (%) | 100 | 104 |
| Antibody alone | % HMW | 3.5 | 12.1 |
| | Aggregation rate (% HMW/h) | — | 0.4 |

### Example 19

### Preparation of a Freeze-Dried Combination of the Antibody and the Polymer P2 in a 1:1 Molar Ratio and Further Extemporaneous Reconstitution with Water for Injection

Example 14 was reproduced by another entity (B) with the same protocol.

### Stage 1: Preparation of the P2 Solution

7.564 g of water for injection was added to 0.438 g of a polymer P2 solution at 71.0 mg/g: the P2 polymer concentration was 3.88 mg/g. The solution was maintained under moderate stirring (15 rpm) for 5 minutes at room temperature.

### Stage 2: Preparation of the Antibody Solution

A frozen solution of the Antibody primary formulation at 42 mg/ml of example 13 was thawed at ambient temperature.

### Stage 3: Preparation of the Liquid Combination of the Antibody and the Polymer P2 in a 1:1 Molar Ratio

6.730 g of the Antibody solution was slowly added onto 7.292 g of the prepared P2 solution. The flask was stirred for 10 minutes at 4° C. on a roller stirrer. Then, 106.8 mg of sucrose was added and the flask was stirred for 10 minutes at 4° C. on a roller stirrer. The obtained solution contained 20.01 mg/g of the Antibody, 2.00 mg/g of the Polymer P2, and 17.56 mg/g of sucrose.

### Stage 4: Freeze-Drying of the Antibody/Polymer P2 Combination in the Presence of Sucrose

The solution of stage 3 was distributed within 10 vials containing approximately 1.2 ml and lyophilized during 76 hours.

### Stage 5: Reconstitution of the Antibody/Polymer P2 Formulation

Approximately 0.197 g of water for injection was slowly added onto each Antibody/Polymer P2 freeze-dried formulation. Each vial was stirred for 10 minutes at 4° C. on a roller stirrer. The obtained clear solution contained approximately 100.0 mg of Antibody, 10.0 mg of polymer P2, and 87.8 mg of sucrose per mL. See FIG. 9.

| Formulation | Assays | T0 | 24 h at RT |
|---|---|---|---|
| Antibody/P2 | Protein recovery (%) | 99 | 105 |
| 1:1 molar ratio | HMW (%) | 4.5 | 8.3 |
| | Aggregation rate (% HMW/h) | — | 0.2 |
| Antibody alone | Protein recovery (%) | 100 | 104 |
| (example 18) | HMW (%) | 3.5 | 12.1 |
| | Aggregation rate (% HMW/h) | — | 0.4 |

The combination of the P2 polymer and the Antibody in a 1:1 molar ratio prevented the Antibody from aggregation.

### Other Sequences Disclosed:

| SEQ | | |
|---|---|---|
| ID | | |
| NO. | Description | Amino Acid Sequence |
| 23 | Constant Heavy Domain | MEFGLSWVFLVAILKGVQCEVQLVESGGVVVQPG |
| | (IGHG1, Genbank | GSLRLSCAASGFTFDDYAMHWVRQAPGKGLEWVS |
| | accession number Q569F4) | LISWDGGSTYYADSVKGRFTISRDNSKNSLYLQMN |
| | | SLRAEDTALYYCATRGGYSTAGFDYWGQGTLVTV |
| | | SSASTKGPSVFPLAPSSKSTSGGTAALGCLVKDYFP |
| | | EPVTVSWNSGALTSGVHTFPAVLQSSGLYSLSSVVT |
| | | VPSSSLGTQTYICNVNHKPSNTKVDKKVEPKSCDKT |
| | | HTCPPCPAPELLGGPSVFLFPPKPKDTLMISRTPEVT |
| | | CVVVDVSHEDPEVKFNWYVDGVEVHNAKTKPREE |
| | | QYNSTYRVVSVLTVLHQDWLNGKEYKCKVSNKAL |
| | | PAPIEKTISKAKGQPREPQVYTLPPSRDELTKNQVSL |
| | | TCLVKGFYPSDIAVEWESNGQPENNYKTTPPVLDS |
| | | DGSFFLYSKLTVDKSRWQQGNVFSCSVMHEALHN |
| | | HYTQKSLSLSPGK |
| 24 | Constant Light Domain | MDMRVPAQLLGLLLLWFPGVRCDIQMTQSPSSLSA |
| | (IGKC, Genbank accession | SVGDRVTITCRASQGIRNDLGWYQQKPGKAPKRLIF |
| | number Q502W4) | AASSLQSGVPSRFSGSGSGTEFTLTINSLQPEDFATY |
| | | YCLQYNSYPRTFGQGTKVEIKRTVAAPSVFIFPPSDE |
| | | QLKSGTASVVCLLNNFYPREAKVQWKVDNALQSG |
| | | NSQESVTEQDSKDSTYSLSSTLTLSKADYEKHKVY |
| | | ACEVTHQGLSSPVTKSFNRGEC |
| 25 | HINGE IgG1 1 | ELLGG |
| 26 | HINGE2 IgG1 2 | MISRT |

What is claimed is:

1-80. (canceled)

81. A stable antibody formulation comprising:
a bispecific anti-IL-4/anti-IL-13 antibody or an antigen binding fragment thereof comprising:
a light chain of the formula N-VL1-linker-VL2-CL-C, wherein CL is a light chain constant domain of an antibody, and
a heavy chain of the formula N-VH1-linker-VH2-CH1-C, wherein CH1 is a heavy chain constant domain of an antibody,
wherein VL1 and VH1 form an outer (N-terminal) IL-13 antigen binding domain, and wherein VL2 and VH2 form an inner (C-terminal) IL-4 antigen binding domain;
a polyaminoacid consisting of glutamic acid or aspartic acid or both with an average degree of polymerization between 25 and 200, and randomly grafted with 1 to 13% mol/mol of Vitamin E; and
a cryoprotectant;
wherein the molar ratio of the bispecific antibody or antigen binding fragment thereof versus the polyaminoacid is between 1:0.25 and 1:2.5, and
wherein the concentration of salt in the formulation is less than 50 mM.

82. The formulation of claim 81, further comprising a buffering system, wherein the formulation comprises a pH of about 7.

83. The formulation of claim 81, wherein
VL1 comprises the CDR sequences of RASESVDSYGQSYMH (CDR1; SEQ ID NO: 8), LASNLES (CDR2; SEQ ID NO: 9), and QQNAEDSRT (CDR3; SEQ ID NO: 10),
VH1 comprises the CDR sequences of GFSLTDSSIN (CDR1; SEQ ID NO: 11), DGRID (CDR2; SEQ ID NO: 12), and DGYFPYAMDF (CDR3; SEQ ID NO: 13),
VL2 comprises the CDR sequences of HASQNIDVWLS (CDR1; SEQ ID NO: 14), KASNLHTG (CDR2; SEQ ID NO: 15), and QQAHSYPFT (CDR3; SEQ ID NO: 16); and
VH2 comprises the CDR sequences of GYSFTSYWIH (CDR1; SEQ ID NO: 17), IDPSDGETR (CDR2; SEQ ID NO: 18), and LKEYGNYDSFYFDV (CDR3; SEQ ID NO: 19).

84. The formulation of claim 81, wherein
VL1 comprises the CDR sequences of RASESVDSYGQSYMH (CDR1; SEQ ID NO: 8), LASNLES (CDR2; SEQ ID NO: 9), and QQNAEDSRT (CDR3; SEQ ID NO: 10),
VH1 comprises the CDR sequences of GFSLTDSSIN (CDR1; SEQ ID NO: 11), DGRID (CDR2; SEQ ID NO: 12), and DGYFPYAMDF (CDR3; SEQ ID NO: 13),
VL2 comprises the CDR sequences of HASQNIDVWLS (CDR1; SEQ ID NO: 14), KASNLHTG (CDR2; SEQ ID NO: 15), and QQAHSYPFT (CDR3; SEQ ID NO: 16); and
VH2 comprises the CDR sequences of GYSFTSYWIH (SEQ ID NO: 20), IDASDGETR (SEQ ID NO: 21), and LKEYGNYDSFYFDV (SEQ ID NO: 22).

85. The formulation of claim 81, wherein
VL1 comprises the amino acid sequence of SEQ ID NO: 1;
VH1 comprises the amino acid sequence of SEQ ID NO: 2;
VL2 comprises the amino acid sequence of SEQ ID NO: 3; and
VH2 comprises the amino acid sequence of SEQ ID NO: 4 or SEQ ID NO: 5.

86. The formulation of claim 81, wherein the linker comprises the amino acid sequence of SEQ ID NO: 6.

87. The formulation of claim 81, wherein the heavy chain further comprises a Fc domain of an antibody.

88. The formulation of claim 87, wherein the heavy chain comprises the formula N-VH1-linker-VH2-CH1-CH2-CH3-C, wherein CH2-CH3 is the Fc domain of an antibody.

89. The formulation of claim 81,
wherein CH1 comprises the amino acid sequence of SEQ ID NO: 23; and
wherein CL comprises the amino acid sequence of SEQ ID NO: 24.

90. The formulation of claim 81, wherein the bispecific antibody or antigen binding fragment thereof is a humanized IgG4 bispecific antibody or antigen binding fragment thereof.

91. The formulation of claim 81, wherein the concentration of bispecific antibody or antigen binding fragment is about 100 mg/mL or less.

92. The formulation of claim 81, wherein the polyaminoacid comprises a nominal degree of polymerization of about 50 to about 100.

93. The formulation of claim 81, wherein the polyaminoacid is randomly grafted with about 10% mol/mol of Vitamin E.

94. The formulation of claim 81, wherein the concentration of polyaminoacid is about 5 mg/ml to about 10 mg/mL.

95. The formulation of claim 81, wherein the cryoprotectant concentration is about 30 mg/kg to about 120 mg/g.

96. The formulation of claim 81, wherein the cryoprotectant concentration is about 1% to about 10% (w/v).

97. The formulation of claim 81, wherein the cryoprotectant comprises sucrose or mannitol.

98. The formulation of claim 82, wherein the buffering system comprises at least two buffers.

99. The formulation of claim 82, wherein the buffering system concentration is about 10 mM.

100. The formulation of claim 82, wherein the buffering system comprises Tris buffer at a concentration of about 3.7 mM or a phosphate buffer at a concentration of about 6.3 mM.

101. The formulation of claim 81, wherein the formulation further comprises a surfactant.

102. The formulation of claim 81, wherein the formulation further comprises a stabilizing agent.

103. The formulation of claim 102, wherein the stabilizing agent comprises proline or mannitol.

104. The formulation of claim 81, wherein the formulation is a liquid formulation.

105. The formulation of claim 81, wherein the formulation is a lyophilized formulation.

106. The formulation of claim 81,
wherein the concentration of the bispecific antibody or an antigen binding fragment thereof is about 100 mg/ml;
wherein the bispecific antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;
wherein the concentration of the polyaminoacid is about 10 mg/mL;
wherein the polyaminoacid has a nominal degree of polymerization of 100, and wherein the polyaminoacid comprises about 10% mol/mol of Vitamin E grafted to the polymer.

107. The formulation of claim 81,
wherein the concentration of the bispecific antibody or an antigen binding fragment thereof is about 100 mg/ml;
wherein the bispecific antibody or antigen binding fragment thereof comprises a heavy chain variable region comprising the amino acid sequences of SEQ ID NOs: 2 and 4, and a light chain variable region comprising the amino acid sequences of SEQ ID NOs: 1 and 3;
wherein the concentration of the polyaminoacid is about 5 mg/mL;
wherein the polyaminoacid has a nominal degree of polymerization of 50, and wherein the polyaminoacid comprises about 10% mol/mol of Vitamin E grafted to the polymer.

108. The formulation of either claim 106 or claim 107, wherein the cryoprotectant comprises about 50 mg/g of sucrose.

109. The formulation of either claim 106 or claim 107, wherein the cryoprotectant comprises about 90 mg/g of sucrose.

110. The formulation of either claim 106 or claim 107, further comprising:
about 0.2% (w/v) polysorbate 80;
about 5% (w/v) sucrose;
about 3% (w/v) mannitol; and
a buffering system comprising a Tris buffer concentration of about 3.7 mM and a phosphate buffer concentration of about 6.3 mM, wherein the buffering system concentration is about 10 mM, and
wherein the formulation comprises a pH of about 7.

111. The formulation of either claim 106 or claim 107, further comprising:
about 0.2% (w/v) polysorbate 80;
about 5% (w/v) sucrose;
about 3% (w/v) proline; and
a buffering system comprising a Tris buffer concentration of about 3.7 mM and a phosphate buffer concentration of about 6.3 mM, wherein the buffering system concentration is about 10 mM, and
wherein the formulation comprises a pH of about 7.

112. A method for treating an allergic disease, cancer, asthma, a disease associated with abnormal production of IL-4 or IL-13 or both, or a disease associated with an elevated TH-2 mediated response comprising administering to a subject in need thereof the formulation of claim 81.


SEQUENCE LISTING (26 sequences)

SEQ ID NO: 1 - protein, 111 aa, Artificial Sequence
  humanized mouse/ mouse VL3 region
DIVLTQSPASLAVSLGQRATISCRASESVDSYGQSYMHWYQQKAGQPPKLLIYLASNLESGVPARFSGSGSRTDFTLTIDPVQAEDAATYYCQQNAEDSRTFGGGTKLEIK

SEQ ID NO: 2 - protein, 118 aa, Artificial Sequence
  humanized mouse/mouse VH2 region
EVQLKESGPGLVAPGGSLSITCTVSGFSLTDSSINWVRQPPGKGLEWLGMIWGDGRIDYADALKSRLSISKDSSKSQVFLEMTSLRTDDTATYYCARDGYFPYAMDFWGQGTSVTVSS

SEQ ID NO: 3 - protein, 108 aa, Artificial Sequence
  humanized mouse/mouse VL1 region
DIQMTQSPASLSVSVGDTITLTCHASQNIDVWLSWFQQKPGNIPKLLIYKASNLHTGVPSRFSGSGSGTGFTLTISSLQPEDIATYYCQQAHSYPFTFGGGTKLEIKR

SEQ ID NO: 4 - protein, 124 aa, Artificial Sequence
  humanized mouse/mouse VH1 region
QVQLQQSGPELVKPGASVKISCKASGYSFTSYWIHWIKQRPGQGLEWIGMIDPSDGETRLNQRFQGRATLTVDESTSTAYMQLRSPTSEDSAVYYCTRLKEYGNYDSFYFDVWGAGTLVTVSSA

SEQ ID NO: 5 - protein, 124 aa, Artificial Sequence
  humanized mouse/mouse VH2 region
QVQLQQSGPELVKPGASVKISCKASGYSFTSYWIHWIKQRPGQGLEWIGMIDASDGETRLNQRFQGRATLTVDESTSTAYMQLRSPTSEDSAVYYCTRLKEYGNYDSFYFDVWGAGTLVTVSSA

SEQ ID NO: 6 - protein, 10 aa, Artificial Sequence
  linker
GGGGSGGGGS

SEQ ID NO: 7 - DNA, 30 bp, Artificial Sequence
  primer
ggaggcggagggtccggaggcggaggatcc

SEQ ID NO: 8 - protein, 15 aa, Artificial Sequence
  hB-B13 VL3 CDR
RASESVDSYGQSYMH

SEQ ID NO: 9 - protein, 7 aa, Artificial Sequence
  hB-B13 VL3 CDR
LASNLES

SEQ ID NO: 10 - protein, 9 aa, Artificial Sequence
  hB-B13 VL3 CDR
QQNAEDSRT

SEQ ID NO: 11 - protein, 10 aa, Artificial Sequence
  hB-B13 VH2 CDR
GFSLTDSSIN

SEQ ID NO: 12 - protein, 5 aa, Artificial Sequence
  hB-B13 VH2 CDR
DGRID

SEQ ID NO: 13 - protein, 10 aa, Artificial Sequence
  hB-B13 VH2 CDR
DGYFPYAMDF

SEQ ID NO: 14 - protein, 11 aa, Artificial Sequence
  h8D4-8 VL1 CDR
HASQNIDVWLS

SEQ ID NO: 15 - protein, 8 aa, Artificial Sequence
  h8D4-8 VL1 CDR
KASNLHTG

SEQ ID NO: 16 - protein, 9 aa, Artificial Sequence
  h8D4-8 VL1 CDR
QQAHSYPFT

SEQ ID NO: 17 - protein, 10 aa, Artificial Sequence
  h8D4-8 VH1 CDR
GYSFTSYWIH

SEQ ID NO: 18 - protein, 9 aa, Artificial Sequence
  h8D4-8 VH1 CDR
IDPSDGETR

SEQ ID NO: 19 - protein, 14 aa, Artificial Sequence
  h8D4-8 VH1 CDR
LKEYGNYDSFYFDV

SEQ ID NO: 20 - protein, 10 aa, Artificial Sequence
  h8D4-8 VH2 CDR
GYSFTSYWIH

SEQ ID NO: 21 - protein, 9 aa, Artificial Sequence
  h8D4-8 VH2 CDR
IDASDGETR

SEQ ID NO: 22 - protein, 14 aa, Artificial Sequence
  h8D4-8 VH2 CDR
LKEYGNYDSFYFDV

SEQ ID NO: 23 - protein, 469 aa, Homo sapiens
MEFGLSWVFLVAILKGVQCEVQLVESGGVVVQPGGSLRLSCAASGFTFDDYAMHWVRQAPGKGLEWVSLISWDGGSTYYADSVKGRFTISRDNSKNSLYLQMNSLRAEDTALYYCATRGGYSTAGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 236 aa, Homo sapiens
MDMRVPAQLLGLLLLWFPGVRCDIQMTQSPSSLSASVGDRVTITCRASQGIRNDLGWYQQKPGKAPKRLIFAASSLQSGVPSRFSGSGSGTEFTLTINSLQPEDFATYYCLQYNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 5 aa, Artificial Sequence
  HINGE IgG1 1
ELLGG

SEQ ID NO: 26 - protein, 5 aa, Artificial Sequence
  HINGE2 IgG1 2
MISRT